(19) United States
(12) Patent Application Publication
(10) Pub. No.: US 2022/0152228 A1
(43) Pub. Date: May 19, 2022

(54) COMPOSITIONS AND METHODS FOR RADIOTHERAPY USING CHELATED RADIOTHERAPEUTIC AGENTS AND NON-TARGET TISSUE BLOCKADE

(71) Applicants: Washington University, St. Louis, MO (US); Cornell University, Ithaca, NY (US)

(72) Inventors: Daniel THOREK, St. Louis, MO (US); Diane ABOU, St. Louis, MO (US); Justin WILSON, Ithaca, NY (US); Nikki THIELE, Ithaca, NY (US)

(73) Assignees: Washington University, St. Louis, MO (US); Cornell University, Ithaca, NY (US)

(21) Appl. No.: 17/432,733

(22) PCT Filed: Mar. 1, 2020
(86) PCT No.: PCT/US2020/020567

§ 371 Date: Aug. 20, 2021

Related U.S. Application Data
(60) Provisional application No. 62/812,565, filed on Mar. 1, 2019.
(60) Provisional application No. 62/827,540, filed on Apr. 1, 2019.

(51) Int. Cl. A61K 51/04; A61K 51/08; A61K 51/10; A61K 31/415; A61K 31/4965; A61K 31/18; A61K 31/496; A61K 31/196; A61K 31/4725; A61K 31/235; A61K 31/4166; A61K 31/4422; A61K 31/365; A61P 39/04; A61P 35/00

(52) U.S. Cl. CPC A61K 51/0482; A61P 35/00; A61K 51/1096; A61K 31/415; A61K 31/4965; A61K 31/18; A61K 31/496; A61K 31/196; A61K 31/4725; A61K 31/235; A61K 31/4166; A61K 31/4422; A61K 31/365; A61P 39/04; A61K 51/088

(57) ABSTRACT

Among the various aspects of the present disclosure is the provision of compositions of isotope-ligand complexes, methods of use thereof, and methods of chelating isotopes. The present disclosure also provides for methods for modulating ion channel transportation of radiopharmaceuticals. For example, the inhibition of radiopharmaceutical transport comprises administering an ion channel transport modulating or inhibiting agent (e.g., a calcium channel inhibitor) in an amount effective to inhibit gastrointestinal uptake.

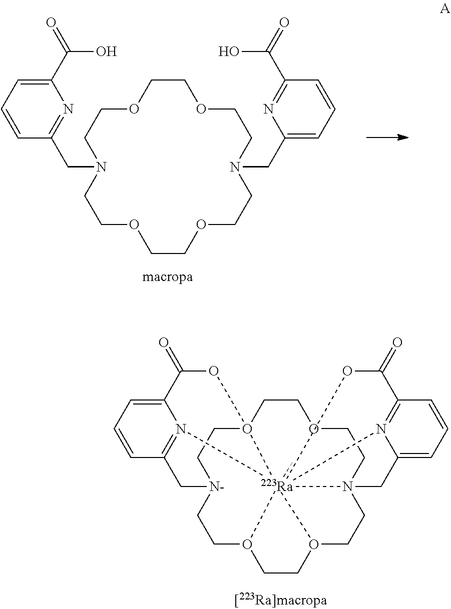

### CROSS-REFERENCE TO RELATED APPLICATIONS

This application claims priority from U.S. Provisional Application Ser. No. 62/827,540 filed on 1 Apr. 2019 and U.S. Provisional Application Ser. No. 62/812,565 filed on 1 Mar. 2019, which are incorporated herein by reference in their entireties.

### STATEMENT REGARDING FEDERALLY SPONSORED RESEARCH OR DEVELOPMENT

This invention was made with government support under CA201035 awarded by the National Institutes of Health. The government has certain rights in the invention.

### MATERIAL INCORPORATED-BY-REFERENCE

Not applicable.

### FIELD OF THE INVENTION

The present disclosure generally relates to methods of improving the delivery of radiopharmaceutical agents using coordination chemistry or combination therapy.

### SUMMARY OF THE INVENTION

Among the various aspects of the present disclosure is the provision of compositions of isotope-ligand complexes and methods of use thereof and methods for chelating isotopes.

One aspect of the present disclosure provides for a method for treating cancer (e.g., bone cancer, metastatic prostate cancer) comprising administering a therapeutically effective amount of an alpha particle-emitting therapeutic agent, wherein the alpha particle-emitting therapeutic agent comprises an alpha particle-emitting isotope-macrocyclic ligand complex. Another aspect of the present disclosure provides for a composition comprising an alpha particle-emitting isotope comprising a radium isotope complexed with a macrocyclic ligand. In some embodiments, the macrocyclic ligand is macropa or a macropa derivative thereof. In some embodiments, the macropa derivative is macropa-NCS. In some embodiments, the alpha particle-emitting isotope is a radium isotope. In some embodiments, the radium isotope is selected from ²²³Ra, ²²⁴Ra, ²²⁵Ra, or ²²⁶Ra. Yet another aspect of the present disclosure provides for method of making an alpha particle-emitting therapeutic agent comprising an alpha particle-emitting isotope complexed with a macrocyclic ligand comprising combining a macrocyclic ligand (e.g., macropa or macropa derivative), Ra(NO₃), RaCl₂, and trimethyl ammonium acetate, wherein the Ra is an Ra isotope and the macrocyclic ligand, Ra(NO₃), RaCl₂, and trimethyl ammonium acetate combined for a period of time sufficient to result in a macrocyclic ligand-radium complex. In some embodiments, the macrocyclic ligand is selected from macropa, macropa-NCS, or a macropa derivative thereof. In some embodiments, the combination is performed at room temperature (about 25° C.), a pH of about 6, for a period of time of about 2 hours. In some embodiments, the radium isotope is selected from ²²³Ra, ²²⁴Ra, ²²⁵Ra, or ²²⁶Ra.

Yet another aspect of the present disclosure provides for a method for treating a subject comprising: administering a therapeutically effective amount of a pharmaceutical composition comprising a macrocyclic ligand selected from a macropa and a macropa derivative to the subject, wherein the macrocyclic ligand is chelated to an alpha particle-emitting radium (Ra) isotope, resulting in a macrocyclic ligand-radium complex. Yet another aspect of the present disclosure provides for a method of minimizing or mitigating exposure or bodily retention of alpha particle-emitting radium (Ra) isotope comprising complexing the alpha particle-emitting Ra isotope in a macrocyclic ligand selected from a macropa and a macropa derivative. In some embodiments, the macrocyclic ligand-radium complex has reduced bone accumulation compared with bone accumulation for a non-chelated radium. In some embodiments, the subject has cancer or metastatic cancer. In some embodiments, the macropa derivative is macropa-NCS. In some embodiments, the alpha particle-emitting Ra isotope is selected from ²²³Ra, ²²⁴Ra, ²²⁵Ra, or ²²⁶Ra. In some embodiments, the macrocyclic ligand-radium complex is (²²³Ra)macropa of formula

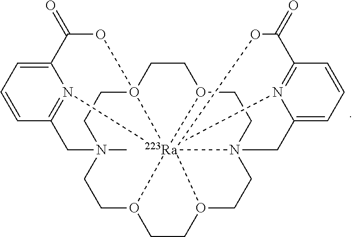

In some embodiments, the macrocyclic ligand is conjugated to a cancer-targeting agent. In some embodiments, the cancer-targeting agent is a monoclonal antibody, a functional fragment of an antibody, a recombinant protein, a single chain variable fragment (scFv), or a peptide. In some embodiments, the macrocyclic ligand-radium complex does not target bone or bone tissue.

Yet another aspect of the present disclosure provides for a pharmaceutical composition comprising a macrocyclic ligand selected from a macropa and a macropa derivative, wherein the macrocyclic ligand is chelated to an alpha particle-emitting radium (Ra) isotope, resulting in macrocyclic ligand-radium complex. In some embodiments, the macropa derivative is macropa-NCS. In some embodiments, the alpha particle-emitting Ra isotope is selected from ²²³Ra, ²²⁴Ra, ²²⁵Ra, or ²²⁶Ra. In some embodiments, the macrocyclic ligand-radium complex is (²²³Ra)macropa of formula

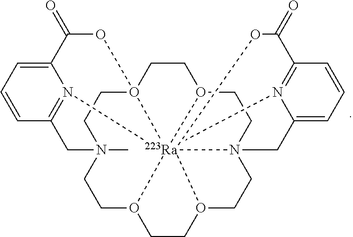

In some embodiments, the macrocyclic ligand is conjugated to a cancer-targeting agent. In some embodiments, the cancer-targeting agent is a monoclonal antibody, a functional fragment of an antibody, a recombinant protein, a single chain variable fragment (scFv), or a peptide.

Yet another aspect of the present disclosure provides for a method of making an alpha particle-emitting therapeutic agent comprising an alpha particle-emitting isotope complexed with a macrocyclic ligand comprising combining a macrocyclic ligand selected from a macropa and a macropa derivative, Ra(NO₃), RaCl₂, and trimethyl ammonium acetate, wherein the alpha particle-emitting isotope is a radium isotope and the macrocyclic ligand, Ra(NO₃), RaCl₂, or trimethyl ammonium acetate are combined for a period of time sufficient to result in a macrocyclic ligand-radium complex. In some embodiments, the macropa derivative is macropa-NCS. In some embodiments, the Ra isotope is selected from ²²³Ra, ²²⁴Ra, ²²⁵Ra, or ²²⁶Ra. In some embodiments, the method comprises conjugating a cancer-targeting agent to the macrocyclic ligand. In some embodiments, the cancer-targeting agent is a monoclonal antibody, a functional fragment of an antibody, a recombinant protein, a single chain variable fragment (scFv), or a peptide. In some embodiments, the combining is performed at about 25° C. and a pH of about 6, for an amount of time sufficient to form a macrocyclic ligand-radium complex, wherein the amount of time sufficient to form a macrocyclic ligand-radium complex is less than an hour or between about 5 minutes and about 2 hours. In some embodiments, the macrocyclic ligand-radium complex does not target bone or bone tissue or is designed to reduce bone or bone tissue uptake. In some embodiments, the macrocyclic ligand is conjugated to a cancer-targeting agent. In some embodiments, the cancer-targeting agent is a monoclonal antibody, a functional fragment of an antibody, a recombinant protein, a single chain variable fragment (scFv), or a peptide.

Among the various aspects of the present disclosure is the provision of methods for modulating ion channel transportation of radiopharmaceuticals. For example, the inhibition of radiopharmaceutical transport comprises administering an ion channel transport modulating or ion channel inhibiting agent (e.g., a calcium channel inhibitor or blocker) in an amount effective to inhibit gastrointestinal uptake. Another aspect of the present disclosure provides for a method of reducing off-target (e.g., gut/gastrointestinal tract, spleen, kidney) uptake of a radiopharmaceutical (e.g., radium-223) in a subject in need thereof comprising administering to the subject an ion channel blocking agent (e.g., calcium channel blocker) in an amount effective to reduce off-target tissue (e.g., gut/gastrointestinal tract, spleen, kidney) localization of the radiopharmaceutical (e.g., radium-223). Yet another aspect of the present disclosure provides for a method of improving radiopharmaceutical (e.g., radium-223) uptake or localization to a target tissue (e.g., bone metastases) of a subject in need thereof comprising administering, to the subject, an ion channel blocking agent (e.g., calcium channel blocker or inhibitor) in an amount effective to increase uptake, targeting, or localization of radiopharmaceutical (e.g., radium-223) in the target tissue (e.g., bone) compared to targeting or localization of radiopharmaceutical (e.g., radium-223) in the target tissue (e.g., bone) without the ion channel blocking agent (e.g., calcium channel inhibiting or blocking agent). In some embodiments, the target tissue is a bone metastasis. In some embodiments, the radiopharmaceutical is a radiopharmaceutical having chemical similarity to calcium or binds to a calcium channel. In some embodiments, the radiopharmaceutical comprises ²²³Ra. In some embodiments, the radiopharmaceutical is ²²³RaCl₂. In some embodiments, the ion channel blocking agent is a calcium channel inhibitor or blocking agent. In some embodiments, the ion channel blocking agent selected from one or more of the group consisting of: Fipronil; Amiloride; Benzamil HCl; AM 92016; SDZ-201 106(+/−); N-Phenylanthranilic acid; Tetrandrine; TMB-8HCl; Dantrolene; Niguldipine HCl; Thapsigargin; and combinations, variants, or analogues thereof. In some embodiments, the ion channel blocking agent is a calcium channel inhibitor selected from amiloride. In some embodiments, the calcium channel inhibitor reduces gastrointestinal uptake of the radiopharmaceutical compared to gastrointestinal uptake of the radiopharmaceutical if no calcium channel inhibitor was administered. In some embodiments, the ion channel inhibiting agent (e.g., calcium channel inhibitor) is a radiopharmaceutical transport inhibiting agent increases radiopharmaceutical (e.g., ²²³Ra) uptake in bone compared to the uptake in bone of a subject not treated with the calcium channel inhibitor.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A-FIG. 1B is a series of macropa chemical structures. Macropa and predicted coordination to radium (A); overlaid DFT modelings of barium-macropa (blue) and radium-macropa (red) displaying metal-ligand coordination bonds of 10 with no noticeable differences (B).

FIG. 2A-FIG. 2C is an image and series of graphs depicting [(²²³Ra)macropa] labeling efficiency report (RL %) observing kinetics and chelator concentration dependence. (A) DGA-coated chromatograms of ²²³RaCl₂ (top) and [(²²³Ra)macropa] (bottom) migrated with NaOH (0.1 M) mobile phase were read utilizing autoradiographic quantification. (B) RL % kinetics defined at room temperature, pH=6, from 5 min to 1 h with varying macropa concentration. (C) Evaluation of minimum macropa concentration to reach RL completion with ²²³Ra after 5 min of reaction, where RL_{50%} was measured at 13 μM and 18 μM was necessary to reach over 80%.

FIG. 3A-FIG. 3E is a series of graphs depicting in vitro stability of [²²³Ra(macropa)]. Size exclusion chromatography (SEC) of (A) (Ba)macropa desorbed at 19 min detected at 280 nm (mAu); and (B) [²²³Ra(macropa)] detected by both UV (dashed line) and gamma counting (blue plain line); (C) Overlay of UV detection (dashed) and gamma counting (plain) of ²²³RaCl₂ mixed in serum proteins; (D) SEC of [(²²³Ra)macropa] mixed in serum proteins; (E) Measured chelated radium following serum protein challenge over 12 days.

FIG. 4A-FIG. 4C is a series of graphs depicting in vivo evaluation of [²²³Ra(macropa)]. (A) ²²³RaCl₂ and (B) [²²³Ra(macropa)] biodistribution was assessed in healthy skeletally mature mice sacrificed at 15 min and 24 h showing differences in splenic; renal and bone uptake. (C) ²²³Ra bone uptake (% IA/g) at 15 min and 24 h p.i. sourced from [²²³Ra(macropa)] (light-15 min; dark blue-24 h) revealed a 10-25 fold lower uptake than free ²²³Ra (grey-15 min/black-24 h) (**P=0.0096; ****P<0.0001). (D) Comparative renal excretion shows a dramatic clearance of ²²³Ra when chelated (*P=0.01); (E) SEC elution of urine identifying intact [²²³Ra(macropa)] complex.

FIG. 5A-FIG. 5C is a series of chemical structures and a graph depicting radium and heavy metals complexes of macropa-β-alanine. (A) Synthesis of macropa-β-alanine and radium complex. (B) Isolated X-ray crystal structure of [[(Ba)macropa]-β-alanine (DMSO)] viewed from the side and top. (C) Potentiometric titration curves for macropa-β-alanine in the presence and absence of 1 equiv of Ca²⁺, Sr²⁺, or Ba²⁺.

FIG. 6A-FIG. 6D is a series of graphs depicting [(²²³Ra)macropa]-β-alanine characterization studies. (A) SEC chromatogram of pure [(²²³Ra)macropa]-β-alanine (dashed line—UV; plain—gamma counting). (B) [(²²³Ra)macropa]-β-alanine challenged in serum protein for 12 days underwent SEC showing protein desorption visible under UV (9-15 mL) and intact [(²²³Ra)macropa]-β-alanine detected at 20 min with gamma counting. (C) [(²²³Ra)macropa]-β-alanine chelated activity was measured through 12 days of serum protein challenge. (D) [(²²³Ra)macropa]-ρ3-alanine radioactive organ distribution 24 h p.i. utilizing naive skeletally mature mice. Significant differences in osseous (*P<0.005), splenic and renal uptakes were noticed with respect to control ²²³RaCl₂.

FIG. 7 is a schematic and image depicting diglycosilamide coated thin layer chromatography. NaOH 10 mM/NaCl; 150 mM solvent; RL=Radiolabeling Efficiency %=Purity %; %[M-L]=% of radiometal-ligand complex.

FIG. 8 is a schematic depicting macropa chelation with ²²³Ra for intravenous injections.

FIG. 9 is a bar graph depicting % IA/g in various samples or organs at 15 minutes and 1 day post-administration of (²²³Ra)macropa and ²²³RaCl₂.

FIG. 10 is a bar graph depicting % IA/g in various samples or organs at 15 minutes and 1 day post-administration of (²²³Ra)macropa and ²²³RaCl₂.

FIG. 11 is a bar graph depicting % IA/g in bone at 15 minutes and 1 day post-administration of (²²³Ra)macropa and ²²³RaCl₂.

FIG. 12A-FIG. 12D is a series of graphs and images depicting gastrointestinal accumulation and excretion of ²²³RaCl₂ and radiobiological effect in the mouse. (A) ²²³Ra dichloride autoradiography of the mouse gastrointestinal organ tract representative subjects. (B) Autoradiographic signal intensity profiles from stomach to cecum displaying the activity migration through the tract (approximately 200 mm/animal) at indicated time points. (C) Upper (stomach), lower (cecum) compartments and complete organ signal quantification, plotted overtime. (D) Immunofluorescent duodenum and colon sections of ²²³RaCl₂ treated (left) and saline control colon (right) samples. For each organ, the upper left corner shows: cell nuclei highlighted with DAPI, in blue; in the upper right corner apoptosis is stained using TUNEL, in purple; lower left depicts cytoskeletal structures using phalloidin, in red overlaid over DAPI; and DNA damage is recognized using γ-H2AX, shown in green.

FIG. 13A-FIG. 13C is a series of schematics and graphs showing in vitro evaluation of ²²³Ra active transport through human duodenum organoids. (A) Schematic of the experimental setup showing human duodenal enteroid monolayers plated on a permeable trans-well scaffold. This organoid recapitulates a functional intestinal barrier suited to examine ²²³Ra gastrointestinal exsorption. (B) ²²³Ra flux evaluated at 37° C. and 4° C. incubation using undifferentiated enteroids shows a temperature-dependent active transport of the radioactivity. (C) ²²³Ra passage through monolayers expressed in percentile comparing differentiated and undifferentiated state of enteroids as a function of time. Differentiated versus undifferentiated enteroids were simultaneously evaluated in order to identify the role of key-ion transporters involved in ²²³Ra transfer. Differentiated duodenal enteroids demonstrated a superior capacity to transport ²²³Ra through the monolayer from basolateral to lumen compartment as compared to the undifferentiated structures (P<0.05). This indicates an ion-channel specific transport of ²²³Ra is mechanistically preferred in the differentiated structures.

FIG. 14A-FIG. 14C is a series of graphs depicting ion-channel inhibitors and activators screen for ²²³Ra flux through caco-2 monolayers and in vivo evaluation of selected compounds. (A) ²²³Ra transport through caco-2 monolayers, from BL to AP compartment, co-incubated with a library of 52 compounds ion-channel inhibitors or activators. Monolayer integrities have been validated post-incubation measuring luminescent flow through of Lucifer yellow permeability (LyP<1.5%). The average radioactive readings (n=3) have been normalized to ²²³Ra flux baseline exempt of treatment. Negative readings indicate inhibition of ²²³Ra transport through monolayers; positive reading indicate flux activation. Amiloride (red) and NS-1619 (green) have been selected for further in vivo validation as inhibitor and activator, respectively. (B) Radioactive organ distribution of healthy male C57/Bl6 mice randomized in 3 groups. Each group (n=6) received a combination treatment with intraperitoneal injection of Amiloride (inhibitor, red) or NS-1619 (activator, green) or saline (control, grey) followed by a clinical dose of ²²³RaCl₂ 1 h post-drug administration. Organs have been harvested, weighed and counted at 15 min, 1 h and 4 h post ²²³Ra treatment. Several organs display significant differences in ²²³Ra uptake overtime. Comparing bone uptake, as early as 15 min, osseous absorption is noticed to be half for NS-1619-treated group as compared to amiloride or the control group. Following blood and kidney clearance at 4 h p.i., ²²³Ra sequestration in bone reveals to be over 1.5 times higher for amiloride-treated group versus control or NS-1619. (C) ²²³Ra activity uptake in bones at 15 min (% IA/g) of control and amiloride-treated group are twice as high as compared to NS-1619 one; this difference is further accentuated for amiloride-treated group 4 h p.i., for whom the bone uptake is significantly higher as compared to the two other groups. The upper GI radioactive uptake (combined % IA of stomach, duodenum, jejunum) suggests a lower radioactive content for amiloride-treated animal 15 min p.i. while the NS-1619 group shows a significantly higher ²²³Ra accumulation 1 h p.i. than the other groups. No difference is noticed 4 h p.i.

Interestingly kidney uptake (% IA) a delayed excretion of ²²³Ra is noticed 4 h p.i. for the amiloride-treated group.

FIG. 15 is a schematic depicting detailed p values and statistics corresponding to FIG. 3B-FIG. 3C.

FIG. 16A-FIG. 16C is a series of images and graphs depicting the toxicity of combination treatment ²²³RaCl₂ and Amiloride versus controls. (A) 4 groups of animals were randomized into non-treated control or treated with: ²²³Ra alone; Amiloride alone and the combination treatment ²²³Ra/Amiloride. Blood chemistry diagnostic profile was conducted for each group evaluating the concentration of Alb.: Albumine (g/L); ALP: Alkaline Phosphatase (U/L); ALT: Alanine Amino Transferase (U/L); AMY: Amyloride (U/L); TBu: Total Bilirubin (μmol/L); BUN: Blood Urea Nitrogen (mmol/L); Ca: Calcium (mmol/L); PHOS: Phosphates (mmol/L); CRT: Creatinine (μmol/L); Glu: Glucose (mmol/L); Na: sodium (mmol/L); K: Potassium (mmol/L); TP: Total Protein (g/L); Glob: Glogulin (g/L). Blood samples were retrieved from day 1 to day 19 post-treatment. Arbitrary units are expressed U. ALP and Creatine present significant concentration drop over 20 days as compared to the control cohort. However, no profile difference was found comparing Radium and Radium/Amiloride cohorts. The combination treatment with Amiloride does not present any toxicity. (B) Kidney physiology was analyzed for each treated group day 20 post-treatment. Kidneys were stained using: Hematoxylin & Eosine (H&E); Masson's Trichrome and Periodic Acid-Schiff stain (PAS). No morphological differences were noticed between each cohorts showing absent kidney toxicity following treatments. (C) Weight monitoring of animals over 20 days following treatment. Weight gain was observed in all treated group. Post-mortem, 20 days following treatment, whole legs were gamma-counted comparing Radium-223 alone and the combination treatment, demonstrating a 15% increase in ²²³Ra bone-homing for the cohort receiving the combination treatment (P=0.0283).

FIG. 17 demonstrates the change in the bone uptake of the control (²²³RaCl₂ alone) and the radiopharmaceutical with Amiloride. The difference in the area under the curve for the combination is significantly larger.

### DETAILED DESCRIPTION OF THE INVENTION

The present disclosure is based, at least in part, on the discovery that radiotherapy can be improved by chelating a radiotherapeutic agent with a macrocyclic molecule or by combining with an ion-modulating agent.

Chelated Radiotherapy

The present disclosure is based, at least in part, on the discovery of a new approach for chelating isotopes, such as ²²³Ra. It is presently believed that this was previously not possible, opening up the possibility of developing safer radiopharmaceuticals, such as ²²³Ra labeled antibodies. As such, macropa radium chelation can also be useful, from a safety standpoint, to minimize or mitigate exposure and bodily retention of radium, in the environment, or in industrial exposure as evidenced by the ²²³Ra-macropa having a different soft tissue (e.g., spleen, kidney, gut) distribution than the free radium ion.

Previous attempts to use calixarene-based ligands for aqueous complexation have been unsuccessful due to either failed radiolabeling or poor kinetic stability of the resulting complex. These unsuccessful efforts have led many in the radiopharmaceutical community to believe that the stable chelation of ²²³Ra cannot be achieved.

Targeted radiotherapy using alpha particle emitters is of emerging commercial interest in oncology. ²²³Ra is the first alpha particle emitting therapy to be approved for clinical use in human cancer patients. The ability to chelate and target ²²³Ra and ²²⁴Ra would be of considerable interest. Chelating agents are needed to make ²²³Ra or ²²⁴Ra monoclonal antibody (mAb) conjugates, which can be used for tumor-specific targeting of the alpha particle emitting Radium. The use of mAB conjugates has the potential to deliver a smaller dose of an alpha particle emitter to a targeted tissue or tumor location, resulting in less toxicity to off-target sites or surrounding tissues. Currently this type of radiotherapy is limited to using Actinium-225 and Thorium-227 as suitable chelating agents.

This disclosure relates to the chemical complexation of isotopes of an element (e.g., radium) with a macrocyclic chelator (macropa) and derivatives of that chelator (macropa-NCS) for the chelation of the element. Radium isotopes (e.g., ²²³Ra, ²²¹Ra, ²²⁴Ra, ²²⁵Ra) are alpha particle emitting isotopes; medical applications primarily utilize the ²²³Ra and ²²⁴Ra isotopes. ²²³Ra dichloride (Xofigo) citrate, delivered as an ion, is an FDA approved and commercially available therapy for metastatic prostate cancer. The bare ion naturally targets sites of bone remodeling (e.g., bone metastases) and thus far the therapeutic targeting has been limited to these sites. The general therapeutic potential of radium isotopes and other alpha particle emitting isotopes could be expanded by targeting the isotopes directly to tumors or other sites of metastases besides the bone. A suitable chelating agent is needed to achieve this targeting; for example, a chelating agent can be used to conjugate an alpha particle emitting isotope to a tumor-targeting vector (e.g., a monoclonal antibody).

As disclosed herein (see e.g., Example 1) demonstrated for the first time, is the stable complexation of ²²³Ra with a ligand. This complexation enables control of the distribution of the therapeutic ²²³Ra element. It has been shown that ²²³Ra can be labeled into a macropa chelator efficiently under mild conditions, and that the complex is stable after administration to animal models. Compared to the bare ion (Xofigo or ²²³RaCl₂) formulation, negligible bone uptake was observed, and the (²²³Ra)macropa complex (the complex of macropa and ²²³Ra can also be denoted by brackets, e.g., [(²²³Ra)macropa]) is quantitatively excreted via renal filtration. As described herein, quality control metrics have also been developed for the purposes of evaluating (²²³Ra)macropa purity and efficiency of labeling. (²²³Ra)macropa may potentially be used in radiolabeling experiments against cancer specific targets.

Radiotherapeutic Agents

As described herein a radiotherapeutic agent, such as an alpha particle-emitting isotope can be complexed in a macrocyclic ligand. The compositions and methods as described herein utilize alpha particle emitters or alpha particle-emitting isotopes complexed with chelation agents (e.g., a macrocyclic ligand) for use in cancer therapy. Alpha emitter radiation therapy uses a radioactive substance that gives off a type of high-energy radiation called an alpha particle to kill cancer cells. The radiotherapeutic agent is injected into a vein, travels through the blood, and collects in certain tissues in the body, such as areas of bone with cancer. This type of radiation may cause less damage to nearby healthy tissue. As an example, alpha emitter radiation therapy is used to treat prostate cancer that has spread to the bone (e.g., metastatic prostate cancer or castration resistant metastatic prostate cancer), and it is being studied in the treatment of other types of cancer.

A radiotherapeutic agent, such as alpha particle emitters, can be any radioactive material known to emit alpha particles and capable of being incorporated into chelation complexes. Alpha particle emitters can be, for example, ²⁰⁹Bi, ²¹¹Bi, ²¹²Bi, ²¹³Bi, ²¹⁰Po, ²¹¹Po, ²¹²Po, ²¹⁴Po, ²¹¹Po, ²¹⁶Po, ²¹¹Po, ²¹⁵At, ²¹⁷At, ²¹⁸At, ²¹⁸Rn, ²¹⁹Rn, ²²⁰Rn, ²²²Rn, ²²⁶Rn, ²²¹Fr, ²²³Ra, ²²⁴Ra, ²²⁵Ra, ²²⁶Ra, ²²⁵Ac, ²²⁷Ac, ²²⁷Th, ²²⁸Th, ²²⁹Th, ²³⁰Th, ²³²Th, ²³¹Pa, ²³³U, ²³⁴U, ²³⁵U, ²³⁶U, ²³⁸U, ²³⁷Np, ²³⁸Pu, ²³⁹Pu, ²⁴⁰Pu, ²⁴⁴Pu, ²⁴¹Am, ²⁴⁴Cm, ²⁴⁵Cm, ²⁴⁸Cm, ²⁴⁹Cf, or ²⁵²Cf. More specifically, the alpha particle-emitter can be ²²³Ra, ²²⁴Ra, ²²⁵Ra, or ²²⁶Ra.

Macrocyclic Ligand

The compositions and methods as described herein utilize alpha particle emitters complexed with chelation agents for use in cancer therapy. The chelation agent can be a macrocyclic ligand.

A macrocyclic ligand can be a macrocycle with a ring size of at least nine (including all hetero atoms) and three or more donor sites. Classic examples are crown ethers and porphyrins. Macrocyclic ligands exhibit particularly high affinity for metal ions.

As described herein, macrocyclic ligands can be macropa or a macropa derivative, or functionalized to be a targeted derivative thereof (see e.g., Scheme 1, Scheme 2, Scheme 3).

Coordination Chemistry

As described herein, isotopes can be complexed with a macrocyclic ligand, such as macropa or a macropa derivative (see e.g., Scheme 1, Scheme 2), Scheme 3.

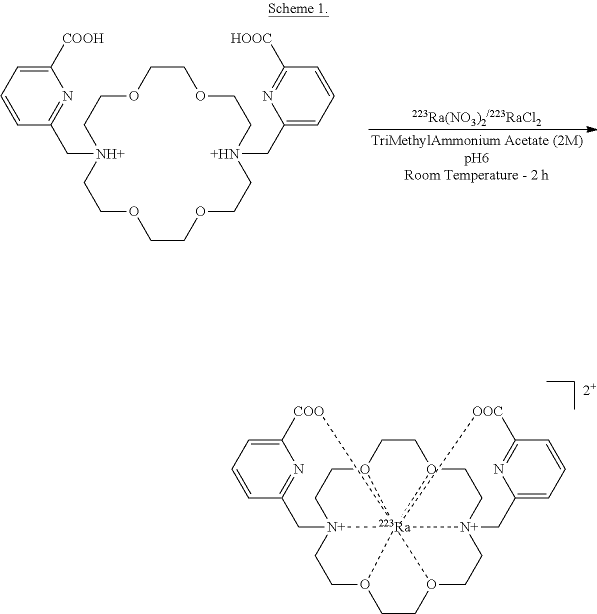

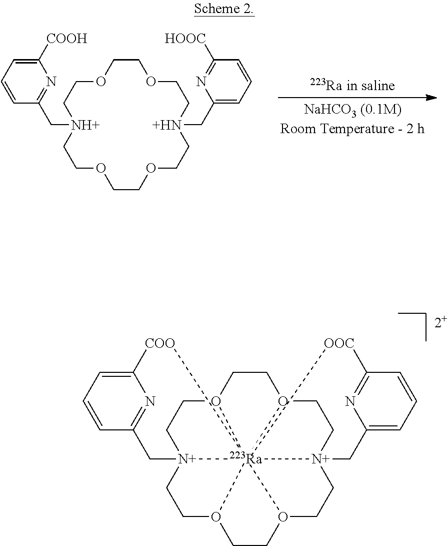

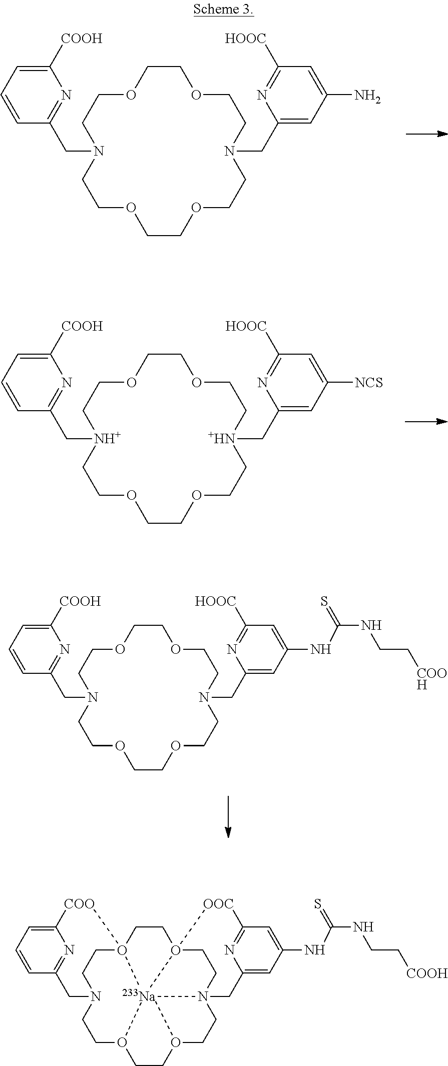

Coordination chemistry can be used herein to generate compounds that have a central atom (often metallic) surrounded by molecules or anions, known as ligands. The ligands can be attached to the central atom by dative bonds, also known as coordinate bonds, in which both electrons in the bond are supplied by the same atom on the ligand.

Cancer-Targeting Agent

As described herein, a macrocyclic ligand can be functionalized and also incorporate a radiotherapeutic agent. One aspect of the present disclosure provides for compositions comprising an alpha particle emitting isotope complexed with a macrocyclic ligand functionalized with a cancer-targeting agent and methods of use thereof. The cancer-targeting agent can be any agent capable of targeting a cancer cell or tumor that can be bound by the macrocyclic ligand. The cancer-targeting agent can enable delivery of the alpha particle to specific sites in a subject (e.g., to a tumor or site of metastasis), increasing therapeutic efficacy and limiting toxicity to non-target tissue. For example, a cancer-targeting agent can be an antibody comprising an epitope that binds to a tumor- or cancer-specific antigen. A tumor- or cancer-specific antigen can be alphafetoprotein, BAGE, beta-catenin-m, beta-Actin/4/m, carcinoembryonic antigen, CA-125, CEA, GAGE, GD2, GD3, globo-H, GM2, Gp100, HER-2/neu, HLA-A2-R170J, HSP70-2/m, livin, mammaglobin-A, MUC-1, myosin/m, epithelial tumor antigen, tyrosinase, melanoma-associated antigen, Melan-A/Mart-1, NY-ESO-1, PSA, SSX, sTn, survivin, tyrosinase, or abnormal products of ras or p53.

Therapeutic Methods for Chelator-Complexed Radioistope

Also provided is a process of treating cancer in a subject in need of administration of a therapeutically effective amount of an alpha particle-emitting therapeutic agent (e.g., a chelator complexed to an alpha-particle-emitting isotope, such as a radium isotope), so as to substantially inhibit cancer or metastasis, slow the progress of cancer or metastasis, or limit the development of cancer or metastasis.

Methods described herein are generally performed on a subject in need thereof. A subject in need of the therapeutic methods described herein can be a subject having, diagnosed with, suspected of having, or at risk for developing cancer. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans. For example, the subject can be a human subject.

Generally, a safe and effective amount of an alpha particle-emitting therapeutic agent is, for example, that amount that would cause the desired therapeutic effect in a subject while minimizing undesired side effects. In various embodiments, an effective amount of an alpha particle-emitting therapeutic agent described herein can substantially inhibit cancer, slow the progress of cancer, or limit the development of cancer.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of an alpha particle emitting therapeutic agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to substantially inhibit cancer, slow the progress of cancer, or limit the development of cancer.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of an alpha particle emitting therapeutic agent can occur as a single event or over a time course of treatment. For example, an alpha particle emitting therapeutic agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for cancer.

An alpha particle-emitting therapeutic agent can be administered simultaneously or sequentially with another agent, such as an antibiotic, an anti-inflammatory, or another agent. For example, an alpha particle-emitting therapeutic agent can be administered simultaneously with another agent, such as an antibiotic or an anti-inflammatory. Simultaneous administration can occur through administration of separate compositions, each containing one or more of an alpha particle-emitting therapeutic agent, an antibiotic, an anti-inflammatory, or another agent.

Combination Radiotherapy and Ion-Channel Modulating Agents

The present disclosure is based, at least in part, on the discovery that the addition of ion channel modulating agents can reduce the non-target tissue (e.g., gastrointestinal, spleen, kidney) uptake of a radiopharmaceutical and increase the uptake of a radiopharmaceutical in the bone. ²²³Ra dichloride (known under the brand name Xofigo, Bayer Healthcare pharmaceuticals) is the first FDA-approved alpha particle emitting pharmaceutical. In the same chemical series as calcium, the isotope is a bone seeker and localizes to sites of remodeling when injected intravenously. This enables it to label sites of bone metastases, and it is currently approved for prostate cancer therapy for patients with bone metastasis. Approximately half of the activity that is administered is rapidly transited via the small bowel into the intestines. The mechanism of action for this process is not understood, however it is known to lead to treatment complications, as well as to the reduction of on-target dose (e.g., the dose intended to target the tumor site). In vitro cell-based and in vivo mouse-based assays were conducted to determine if this process is biologically active and pharmacologically inhibitable. The studies described herein have revealed that a class of ion channel blockers can be used to block uptake of ²²³Ra into the gastrointestinal tract, reducing localization there and thus increasing bone targeting. Additionally, the lead compound that has been investigated is the approved blood pressure medication amiloride.

This drug (amiloride) or other ion channel blockers could be used with ²²³Ra in combination to improve the on-target effect and reduce background effects. This can also be useful in the further drug development of other alpha particle emitting radiotherapeutics in development.

Radiopharmaceuticals

One aspect of the present disclosure provides for modulating uptake of a radiopharmaceutical in a target tissue. A radiopharmaceutical generally refers to any radioactive compound that can be used as a therapeutic or diagnostic agent. For example, a radiopharmaceutical can comprise carbon-11, carbon-14, fluorine-18, gallium-67, gallium-68, indium-111, iodine 1-123, iodine-125, iodine-131, lutetium-177, molybdenum-99, nitrogen-13, radium-223, rubidium-82, samarium-153, or technetium-99m.

As another example, a radiopharmaceutical can be carbon-11 choline, carbon-14 urea, fluorine-18 florbetaben, fluorine-18 florbetapir, fluorine-18 flucicovine, fluorine-18 sodium fluoride, fluorine-18 fludeoxyglucose, fluorine-18 flutemetamol, gallium-67 citrate, gallium-68 dotatate, gallium-68 dotatoc, indium-111 chloride, indium-111 oxyquinoline, indium-111 pentetate, indium-111 pentetreotide, iodine-123 iobenguiane, iodine-123 ioflupane, iodine-123 sodium iodide, iodine-125 serumalbumin, idodine-125 iothalamate, iodine-131 serumalbumin, iodine-131 iobenguane, iodine-131 sodium iodide, lutetium Lu-177 dotatate, molybdenum Mo-99 generator, nitrogen-13 ammonia, radium-223 dichloride, rubidium-82 chloride, samarium-153 lexidronam, technetium-99m bicisate, technetium-99m exametazine, technetium-99m macroaggregated albumin, technetium-99m mebrofenin, technetium-99m medronate, technetium-99m mertiatide, technetium-99m oxidronate, technetium-99m pentetate, technetium-99m pyrophosphate, technetium-99m red blood cells, technetium-99m sestamibi, technetium-99m sodium pertechnetate, technetium-99m succimer, technetium-99m sulfur colloid, technetium-99m tetrofosmin, or technetium-99m tilmanocept.

Ion Channel Transport Modulating Agent

One aspect of the present disclosure provides for modulation of ion-channel transport (e.g., a calcium channel inhibiting or blocking agent). The present disclosure provides for methods of treating bone cancer based on the discovery that modulation or inhibition of ion channels can increase radiopharmaceutical uptake in bone and/or decrease radiopharmaceutical uptake in the gastrointestinal tract.

Ion channel modulating agents can comprise Fipronil; Amiloride; Benzamil HCl; AM 92016; SDZ-201 106(+/−); N-Phenylanthranilic acid; Tetrandrine; TMB-8HCl; Dantrolene; Niguldipine HCl; or Thapsigargin; or combinations, variants, or analogues thereof.

The ion channel modulating agent can be any ion channel modulating agent or pharmaceutically acceptable salt, solvate, or polymorph, thereof.

The term “solvate” is intended to mean a solvate form of a specified compound that retains the effectiveness of such compound. Examples of solvates include compounds of the invention in combination with, for example: water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further includes a stoichiometric or non-stoichiometric amount of water bound by non-covalent intermolecular forces.

Therapeutic Methods for Combination Therapy

Also provided is a process of treating cancer in a subject in need administration of a therapeutically effective amount of a radiopharmaceutical and an ion channel modulating agent, so as to reduce the gastrointestinal uptake of the radiopharmaceutical and/or increase the uptake of the radiopharmaceutical in bone or other organ or tissue of interest.

Methods described herein are generally performed on a subject in need thereof. A subject in need of the therapeutic methods described herein can be a subject having, diagnosed with, suspected of having, or at risk for developing cancer. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans. For example, the subject can be a human subject.

Generally, a safe and effective amount of a radiopharmaceutical and an ion channel modulating agent is, for example, that amount that would cause the desired therapeutic effect in a subject while minimizing undesired side effects. In various embodiments, an effective amount of a radiopharmaceutical and an ion channel modulating agent described herein can substantially inhibit the uptake of radiopharmaceutical in the gastrointestinal tract, increase the uptake of the radiopharmaceutical in bone or other tissue, or inhibit calcium ion channels.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of a radiopharmaceutical and an ion channel modulating agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to can substantially inhibit the uptake of radiopharmaceutical in the gastrointestinal tract, increase the uptake of the radiopharmaceutical in bone or other tissue, or inhibit calcium ion channels.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a radiopharmaceutical and an ion channel modulating agent can occur as a single event or over a time course of treatment. For example, a radiopharmaceutical and an ion channel modulating agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for cancer.

Cancer

Methods and compositions as described herein can be used for the prevention, treatment, or slowing the progression of cancer or metastatic cancer in a subject by administering a complexed alpha-emitting radiotherapeutic. For example, the metastasis can be from or the cancer can be Acute Lymphoblastic Leukemia (ALL); Acute Myeloid Leukemia (AML); Adrenocortical Carcinoma; AIDS-Related Cancers; Kaposi Sarcoma (Soft Tissue Sarcoma); AIDS-Related Lymphoma (Lymphoma); Primary CNS Lymphoma (Lymphoma); Anal Cancer; Appendix Cancer; Gastrointestinal Carcinoid Tumors; Astrocytomas; Atypical Teratoid/Rhabdoid Tumor, Childhood, Central Nervous System (Brain Cancer); Basal Cell Carcinoma of the Skin; Bile Duct Cancer; Bladder Cancer; Bone Cancer (including Ewing Sarcoma and Osteosarcoma and Malignant Fibrous Histiocytoma); Brain Tumors; Breast Cancer; Bronchial Tumors; Burkitt Lymphoma; Carcinoid Tumor (Gastrointestinal); Childhood Carcinoid Tumors; Cardiac (Heart) Tumors; Central Nervous System cancer; Atypical Teratoid/Rhabdoid Tumor, Childhood (Brain Cancer); Embryonal Tumors, Childhood (Brain Cancer); Germ Cell Tumor, Childhood (Brain Cancer); Primary CNS Lymphoma; Cervical Cancer; Cholangiocarcinoma; Bile Duct Cancer Chordoma; Chronic Lymphocytic Leukemia (CLL); Chronic Myelogenous Leukemia (CML); Chronic Myeloproliferative Neoplasms; Colorectal Cancer; Craniopharyngioma (Brain Cancer); Cutaneous T-Cell; Ductal Carcinoma In Situ (DCIS); Embryonal Tumors, Central Nervous System, Childhood (Brain Cancer); Endometrial Cancer (Uterine Cancer); Ependymoma, Childhood (Brain Cancer); Esophageal Cancer; Esthesioneuroblastoma; Ewing Sarcoma (Bone Cancer); Extracranial Germ Cell Tumor; Extragonadal Germ Cell Tumor; Eye Cancer; Intraocular Melanoma; Intraocular Melanoma; Retinoblastoma; Fallopian Tube Cancer; Fibrous Histiocytoma of Bone, Malignant, or Osteosarcoma; Gallbladder Cancer; Gastric (Stomach) Cancer; Gastrointestinal Carcinoid Tumor; Gastrointestinal Stromal Tumors (GIST) (Soft Tissue Sarcoma); Germ Cell Tumors; Central Nervous System Germ Cell Tumors (Brain Cancer); Childhood Extracranial Germ Cell Tumors; Extragonadal Germ Cell Tumors; Ovarian Germ Cell Tumors; Testicular Cancer; Gestational Trophoblastic Disease; Hairy Cell Leukemia; Head and Neck Cancer; Heart Tumors; Hepatocellular (Liver) Cancer; Histiocytosis, Langerhans Cell; Hodgkin Lymphoma; Hypopharyngeal Cancer (Head and Neck Cancer); Intraocular Melanoma; Islet Cell Tumors; Pancreatic Neuroendocrine Tumors; Kaposi Sarcoma (Soft Tissue Sarcoma); Kidney (Renal Cell) Cancer; Langerhans Cell Histiocytosis; Laryngeal Cancer (Head and Neck Cancer); Leukemia; Lip and Oral Cavity Cancer (Head and Neck Cancer); Liver Cancer; Lung Cancer (Non-Small Cell and Small Cell); Lymphoma; Male Breast Cancer; Malignant Fibrous Histiocytoma of Bone or Osteosarcoma; Melanoma; Melanoma, Intraocular (Eye); Merkel Cell Carcinoma (Skin Cancer); Mesothelioma, Malignant; Metastatic Cancer; Metastatic Squamous Neck Cancer with Occult Primary (Head and Neck Cancer); Midline Tract Carcinoma Involving NUT Gene; Mouth Cancer (Head and Neck Cancer); Multiple Endocrine Neoplasia Syndromes; Multiple Myeloma/Plasma Cell Neoplasms; Mycosis Fungoides (Lymphoma); Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms; Nasal Cavity and Paranasal Sinus Cancer (Head and Neck Cancer)’ Nasopharyngeal Cancer (Head and Neck Cancer)’ Neuroblastoma; Non-Hodgkin Lymphoma; Non-Small Cell Lung Cancer; Oral Cancer, Lip or Oral Cavity Cancer; Oropharyngeal Cancer (Head and Neck Cancer); Osteosarcoma and Malignant Fibrous Histiocytoma of Bone; Ovarian Cancer Pancreatic Cancer; Pancreatic Neuroendocrine Tumors (Islet Cell Tumors); Papillomatosis; Paraganglioma; Paranasal Sinus and Nasal Cavity Cancer (Head and Neck Cancer); Parathyroid Cancer; Penile Cancer; Pharyngeal Cancer (Head and Neck Cancer); Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer Renal Cell (Kidney) Cancer; Retinoblastoma; Rhabdomyosarcoma, Childhood (Soft Tissue Sarcoma); Salivary Gland Cancer (Head and Neck Cancer); Sarcoma; Childhood Rhabdomyosarcoma (Soft Tissue Sarcoma); Childhood Vascular Tumors (Soft Tissue Sarcoma); Ewing Sarcoma (Bone Cancer); Kaposi Sarcoma (Soft Tissue Sarcoma); Osteosarcoma (Bone Cancer); Uterine Sarcoma; Sezary Syndrome (Lymphoma); Skin Cancer; Small Cell Lung Cancer; Small Intestine Cancer; Soft Tissue Sarcoma; Squamous Cell Carcinoma of the Skin; Squamous Neck Cancer with Occult Primary, Metastatic (Head and Neck Cancer); Stomach (Gastric) Cancer; T-Cell Lymphoma, Cutaneous; Lymphoma; Mycosis Fungoides and Sezary Syndrome; Testicular Cancer; Throat Cancer (Head and Neck Cancer); Nasopharyngeal Cancer; Oropharyngeal Cancer; Hypopharyngeal Cancer; Thymoma and Thymic Carcinoma; Thyroid Cancer; Thyroid Tumors; Transitional Cell Cancer of the Renal Pelvis and Ureter (Kidney (Renal Cell) Cancer); Ureter and Renal Pelvis; Transitional Cell Cancer (Kidney (Renal Cell) Cancer; Urethral Cancer; Uterine Cancer, Endometrial; Uterine Sarcoma; Vaginal Cancer; Vascular Tumors (Soft Tissue Sarcoma); Vulvar Cancer; or Wilms Tumor.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc., Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, subcutaneous, intranasal, epidural, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Administration

Agents and compositions described herein can be administered according to methods described herein in a variety of means known to the art.

As discussed above, administration can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to ligands, chelators, isotopes, ion-channel modulating agents, or radionuclides. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Radium Chelation for Targeted Alpha Particle Therapy

The following example describes the chemical complexation of radium.

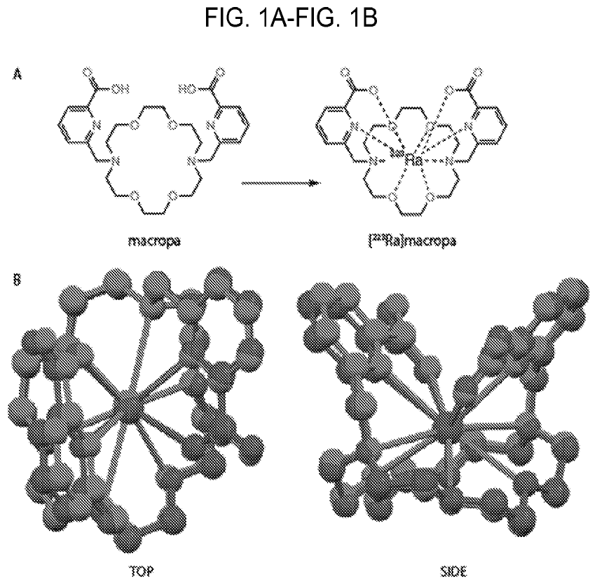

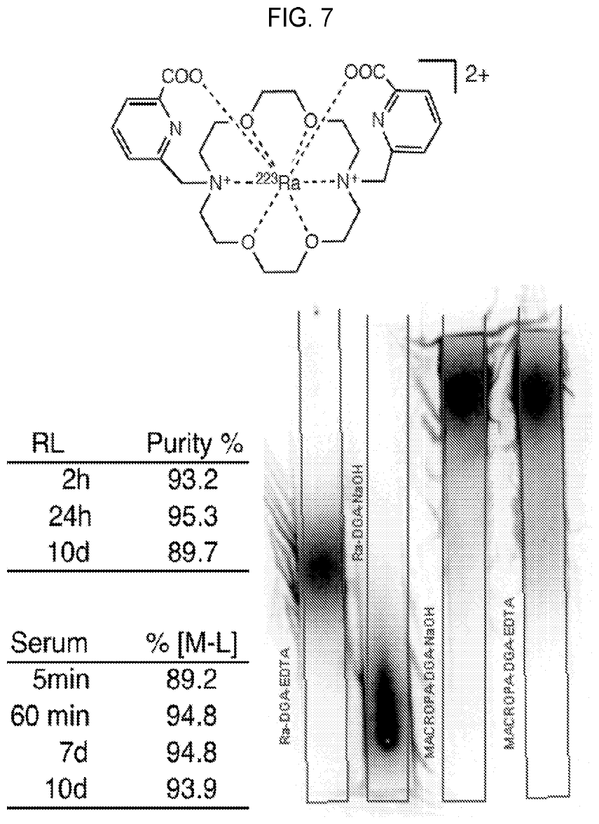

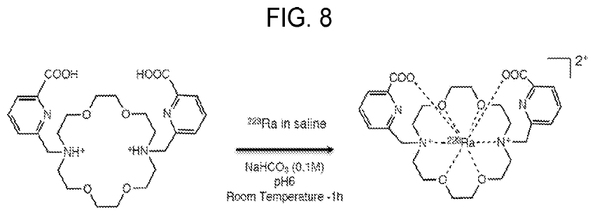

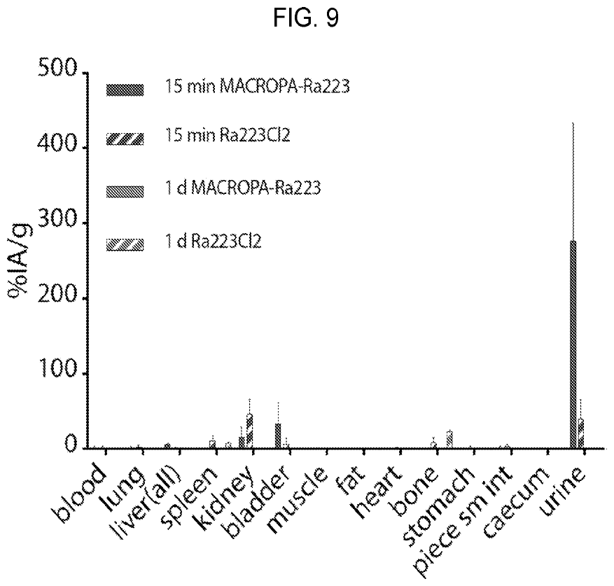

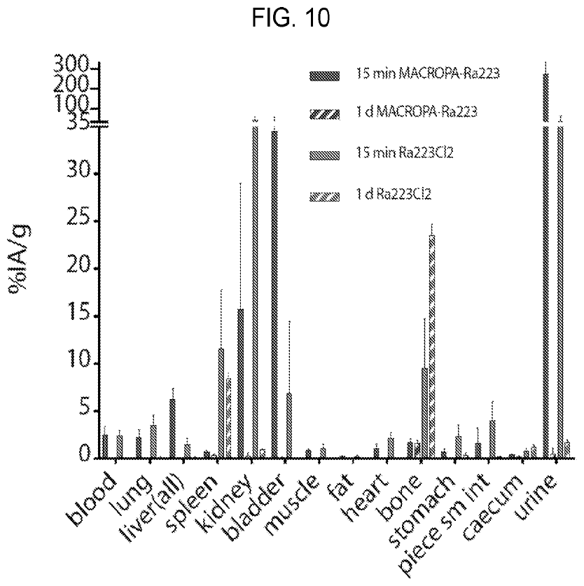

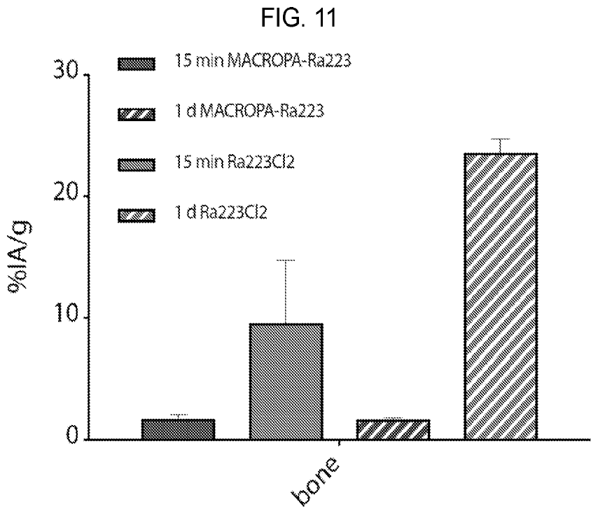

Through the chelation work disclosed herein, it has been demonstrated for the first time the stable complexation of ²²³Ra with a ligand—this enables control of the distribution of the therapeutic ²²³Ra element. It is also shown herein that ²²³Ra can be labeled into a macropa chelate efficiently under mild conditions, and that the complex is stable after administration to animal models (see e.g., FIG. 1, FIG. 7, FIG. 8). Compared to the bare ion (Xofigo) formulation, negligible bone uptake was observed, and the (²²³Ra)macropa complex is quantitatively excreted via renal filtration (see e.g., FIG. 9, FIG. 10, FIG. 11). Quality control metrics have also been developed for the evaluation (²²³Ra)macropa purity and efficiency of labeling. Current studies are being performed on targeted radiolabeling experiments against cancer specific targets.

Targeted internal isotope radiotherapy delivers high and localized treatment doses to disseminated sites of disease while sparing normal surrounding organs from toxic effects. ²²³RaCl₂ is the first approved alpha particle emitting radionuclide for treatment of castrate resistant prostate cancer, exploiting the radium ion's incorporation at bone metastasis sites. While effective, the agent does not integrate directly cancerous tissues. To specifically direct ²²³Ra to cancerous tissues, chelation and functionalization with a cancer-targeting moiety can be performed.

As reported herein, radium chelation utilizing an 18-membered bis-picolinate diazacrown ring, macropa was successfully demonstrated. [²²³Ra(macropa)] complexation was rapidly achieved with a radiolabeling efficiency (RL) >95% at room temperature and pH=6. The complex displayed high stability against serum protein challenges: >90% of ²²³Ra was retained over 12 days. In vivo, [²²³Ra(macropa)] displayed a change in ²²³Ra organ distribution with strikingly low bone accumulation of ²²³Ra in mice when compared to free ²²³RaCl₂. ²²³Ra-macropa was identified excreted intact in urines.

Similarly, macropa conjugated to β-alanine resulted in RL>95% and retained >75% of ²²³Ra over 12 days of challenge. In vivo, [²²³Ra(macropa)]-β-alanine was cleared rapidly and low ²²³Ra absorption in the bone. These results demonstrate the high affinity of macropa chelator with ²²³Ra even when conjugated to an amino-acid. The successful conjugation with β-alanine, provides evidence that further functionalization [(²²³Ra)macropa] can be performed towards cancer-specific targets.

### Introduction

There has been a resurgence of interest in systemic molecular radiotherapy as biochemical approaches to target sites of disease have improved. Recent advances have focused on harnessing alpha particle (α)-emitters for targeted radionuclide therapy. Alpha particles are charged helium nuclei that deposit several MeV of energy along the distance of only several cell diameters to provide potent but selective cytotoxicity. Indeed, a single α-particle traversal through a cell can be lethal. These effects are independent of oxygen concentration or genetic resistance pathways that manifest in conventional external beam X-ray radiotherapy.

²²³RaCl₂ is the first and currently only approved α-emitting radiopharmaceutical, with an indication for men with bone-metastatic castrate resistant prostate cancer. Since its approval in 2013, ²²³RaCl₂ has been used to treat over 18,000 patients with this disease, substantially improving the quality of life for those suffering from bone pain, reducing fracture risk and prolonging overall survival. The ²²³Ra radiometal is employed without a biological targeting vector or chelating agent. ²²³Ra²⁺ is readily incorporated in bones undergoing turnover, including at sites of osseous metastases, where it subsequently decays to irradiate the surrounding malignant tissue. This therapeutic strategy exploits the chemical similarity between the Ra²⁺ ion and the endogenous Ca²⁺ ion. Through its decay to the stable daughter ²⁰⁷Pb, the 4 high-energy, short-range α's of the ²²³Ra decay annihilate disseminated prostate cancer cells, while largely sparing neighboring healthy tissues.

Given the established clinical efficacy, safety, and widespread availability of ²²³Ra in comparison to other α-emitters such as ²²⁵Ac, the implementation of this radiometal in targeted alpha particle therapy (TAT) is very attractive. To effectively use ²²³Ra as a molecularly specific agent, this radiometal must be stably conjugated to a tumor-targeting vector, such as an antibody or peptide, via a bifunctional chelating agent (BFC). An effective BFC must match the coordination chemistry of the radiometal of interest such that the ion remains stably bound to the targeting vector in vivo. Despite decades of interest for biomedical and environmental applications, no effective chelator for ²²³Ra has been identified.

The development of chelators for the stable chelation of Ra²⁺ is challenging. As an s-block ion, its interactions with ligands is predominantly electrostatic in nature. Ra²⁺ is the largest +2 ion in the periodic table (8-coordinate ionic radius=1.48 Å), and thus possesses a low charge-to-ionic radius ratio that gives rise to electrostatic metal-ligand interactions that are substantially weaker than those of the smaller alkaline earth ions. Efforts to chelate Ra²⁺ thus far have almost exclusively been limited to organic-soluble extractants that partition this ion to the organic phase in liquid-liquid separations. Of these extractants, the most common structural motif explored has been that containing a calixarene core. Attempts to use these calixarene-based ligands for aqueous complexation have been unsuccessful due to either failed radiolabeling or poor kinetic stability of the resulting complex. These unsuccessful efforts have led many in the radiopharmaceutical community to believe that the stable chelation of ²²³Ra cannot be achieved.

Recently, it was reported that macropa, an eighteen-membered bis-picolinate diazacrown macrocycle (see e.g., FIG. 1A), forms a complex of high thermodynamic stability with Ba²⁺, the largest non-radioactive +2 ion (eight-coordinate ionic radius=1.42 Å). In aqueous solution at physiological pH, the stability constant (log K′_{ML,pH7.4}) of the [(Ba)macropa] complex is 10.74, rendering macropa the highest-affinity chelator for Ba²⁺ near neutral pH reported to date. It was hypothesized in the present disclosure that macropa may also be effective for the stable chelation of ²²³Ra²⁺ due to similar chemical properties and size with Ba²⁺. Furthermore, macropa has also been utilized to chelate other large radioactive metal ions such as ²²⁵Ac and ^{132/135}La. In this study, the kinetics, stability and in vivo utility of this ligand for generation of novel ²²³Ra targeted therapeutics was investigated.

### Results and Discussion

Macropa is a macrocyclic crown bearing two pendent arms electronic donors—picolinic acids—participating in the coordination of heavy metals (Ca²⁺ Sr²⁺, Ba²⁺). The inner diameter of the macrocyclic cage is favorable to barium coordination; and similarly is suitable for ²²³Ra chelation through 10 coordination sites (see e.g., FIG. 1A). To confirm this hypothesis, DFT modeling of radium-macropa and barium-macropa were computed and metal coordination bond lengths were measured. An overlay of the two metals chelated by macropa are shown in FIG. 1B.

TABLE 1
Interatomic distances (Å) of the metal coordination environments of [M(macropa)]
calculated at the TPSSh/TZVP/LC RECP level of theory.

| | M—N_{PY} | M—N_{PY} | M—O_{COO} | M—O_{COO} | M—O_{C} | M—O_{C} | M—O_{C} | M—O_{C} | M—N_{AM} | M—N_{AM} |
|---|---|---|---|---|---|---|---|---|---|---|
| Ba | 2.829 | 2.831 | 2.625 | 2.628 | 2.956 | 2.957 | 3.023 | 3.025 | 3.09 | 3.095 |
| Ra | 2.897 | 2.895 | 2.7 | 2.7 | 3.015 | 3.016 | 3.052 | 3.057 | 3.133 | 3.137 |

[symbol]

N_{PY} = pyridyl nitrogen atoms, O_{COO} = carboxylate oxygen atoms, O_{C} = crown oxygen atoms, N_{AM} = amine nitrogen atoms. [symbol]
indicates data missing or illegible when filed

Negligible differences in bond lengths were observed when comparing Ra and Ba complex modeling, suggesting that the suitability of Ba(macropa) may translate to chelation of radium (see e.g., FIG. 1C).

To evaluate radium macropa radiolabeling efficiency and kinetics, macropa was synthesized according to established procedures and produced nitrate free ²²³Ra from an ²²⁷Ac/²²⁷Th generator, as previously described.

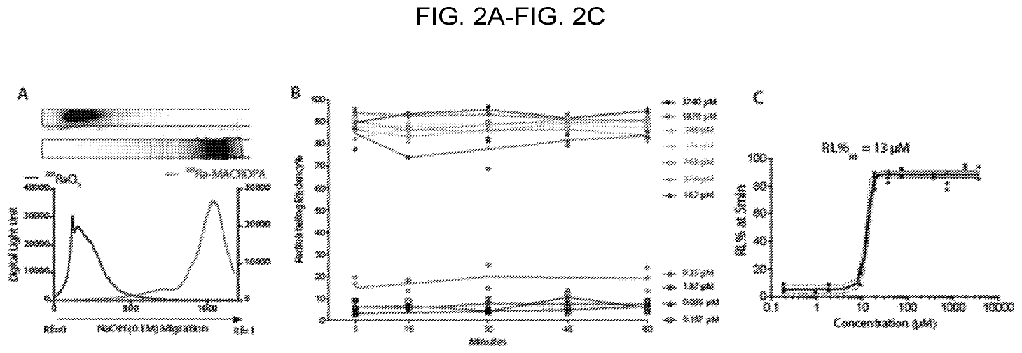

Formation of the complex was monitored by radio-instant thin layer chromatography (radioTLC) at radioisotopic equilibrium. RadioTLC demonstrated a distinct shift in the mobilization of ²²³RaCl₂ (Rf=0) and complexed species' migration (Rf≅1). [²²³Ra(macropa)] was formed rapidly at room temperature, as early as 5 min, and at near-physiological pH 6 (see e.g., FIG. 2). This mild radiolabeling condition is a highly advantageous approach to labeling heat-sensitive biomolecules of interest for potential biomedical applications.

Kinetics of chelation were assessed from 5 min to 1 h post-radioactive ion addition. Similar results were acquired at early and late time points; suggesting an instantaneous chelation to completion of radium (see e.g., FIG. 2B). [²²³Ra(macropa)] radiolabeling efficiency exceeded 95% (n=10) utilizing micromolar concentrations of chelator. This contrasts with the most widely used heavy metal chelator 1,4,7,10-Tetraazacyclododecane-1,4,7,10-tetraacetic acid (DOTA) which does not coordinate radium in similar conditions.

Varying the chelator concentration with constant ²²³Ra activity added (3.7 kBq), a striking difference in RL % was observed. Below 18 μM no or poor complexation was measured; while at greater concentrations, labeling was achieved to almost completion exceeding 80%. In the mM range, RL % was completed over 95%. Correlating RL % versus chelator concentration predicted 50% of the radiolabeling can be completed with 13 μM of macropa. An identical concentration was extrapolated for RL %₅₀ after 1 h, further showing that no difference of efficiency was observed over this time frame. Thus, in aqueous conditions, [²²³Ra(macropa)] is rapidly formed in high yield.

Next, the stability of the complex to serum proteins was determined in vitro, simulating the complex biological environment encountered in vivo. Stability was evaluated for [(²²³Ra)macropa] in human serum proteins at 37° C. over 12 days.

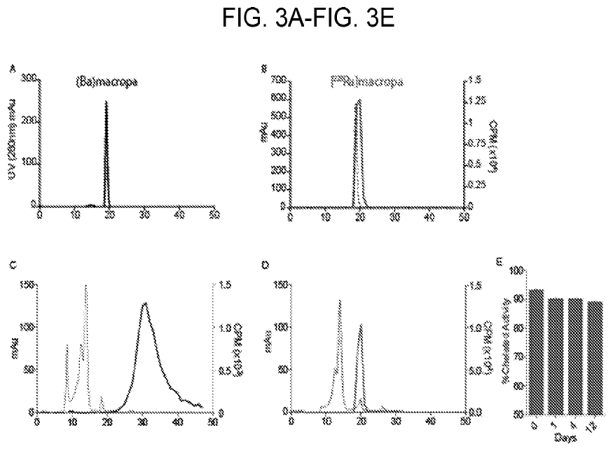

While radium complexation utilizing radioTLC in buffer was successfully assessed (see e.g., FIG. 1A), this analytical technique was not suitable in defining decomplexation of radium in the presence of serum proteins. Alternatively, size exclusion chromatography (SEC) was utilized to identify each species in solution. Combining both detection modalities: UV at 280 nm and gamma counting of fractions, a separation of protein aggregates and macromolecules (˜100 kDa; 5-10 mL) from [(²²³Ra)macropa] (˜1 kDa; 20 mL), from free ion ²²³Ra (>30 mL) was visualized. First, pure (Ba)- and (²²³Ra)-macropa were eluted under matching peaks at 20 mL (see e.g., FIG. 3A, FIG. 3B). Then, free ²²³Ra and [(²²³Ra)macropa] in serum protein mixture were separated from proteins with an unchanged elution peak for both [(²²³Ra)macropa] and ²²³Ra (see e.g., FIG. 3C, FIG. 3D). Chelated activity % was measured integrating gamma counts of macropa peak (20 mL) over the sum counts of all collected fractions (0-47 mL) (see e.g., FIG. 3E). Following 2 h of incubation in serum proteins, both ²²³RaCl₂ (see e.g., FIG. 3C) and [²²³Ra(macropa)] (see e.g., FIG. 3D) presented no registered activity in the early fractions (5-15 mL); suggesting complex and free radium did not present any free affinity with proteins. As such, it can be assumed that in the event of ²²³Ra decomplexation only free radium peak (>30 mL) will be increasing.

Serum protein challenges over 12 days, the course of approximately one ½ life decay of ²²³Ra, demonstrated the high stability of [(²²³Ra)macropa] with only 10% of decomplexation (see e.g., FIG. 3C). This was similarly recapitulated with non-radioactive [(Ba)macropa] challenged with hydroxyapatite, a major constituent of bone matrix that are presently thought to also bind these metals. These results compare favorably with in vitro assessment of other alpha particle targeted radiotherapies that have entered clinical practice. In light of this strong coordination of ²²³Ra with macropa, the biodistribution of the radiocomplex in vivo was evaluated by organ-based radioactive counting assays.

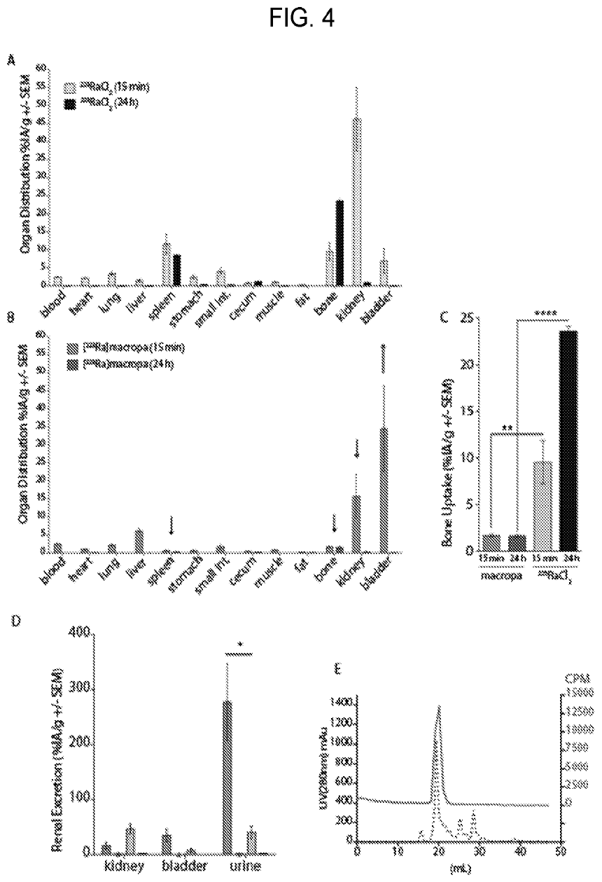

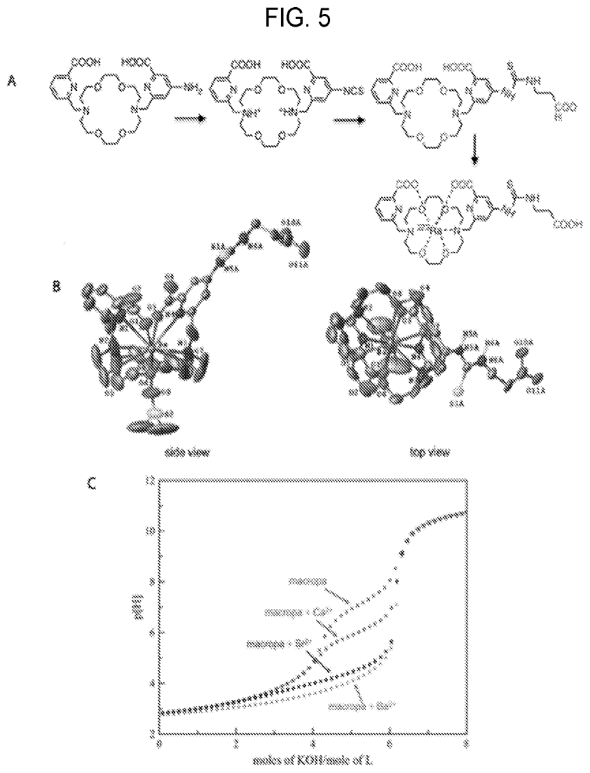

It was previously shown that ²²³RaCl₂ distribution in mice closely resembles that observed in man—with rapid blood clearance and localization to sites of active bone remodeling, with excretion predominantly occurring through the small bowel and kidneys. The in vivo stability of chelated [²²³Ra(macropa)] was assessed through comparison of the approved formulation in rodent models by high sensitivity and quantitative gamma counting. A comprehensive radioactive organ biodistribution was surveyed at 15 min and 24 h post-injection (p.i.) and normalized to tissue weight (see e.g., FIG. 4). At 15 min p.i., a 10-fold lower uptake in the bone was observed for the [(²²³Ra)macropa] administered animals compared to the ²²³RaCl₂ in citrate. The difference increased at 24 h p.i. to 20-fold where [(²²³Ra)macropa] presented 1.6±0.2 percent injected activity per gram (% IA/g) versus 22±1% IA/g for the control group (P<0.0001; FIG. 4B).

The radiometal-ligand complex also shifted the excretion profile markedly, beyond the bone. Stable complexation prevented accumulation in the spleen (previously noted as a site of uptake in the rodent) and the gut. This is significant as gastrointestinal distress is a commonly reported symptom from ²²³RaCl₂ treatment as nearly one-half of the agent is passed via this route. All other organs of interest at 24 p.i. present negligible counts. The overall result is that mice retained less activity in the [(²²³Ra)macropa] group.

The majority of small molecule [(²²³Ra)macropa] is rapidly cleared from the blood by the kidney. At the early time point a 300-fold higher signal of [(²²³Ra)macropa] was measured in an equal volume of urine (sampled from the bladder) than that of ²²³RaCl₂ group. The lower kidney activity at this early time point with increased bladder accumulation indicates that the chelator is quickly passed, in contrast to the longer retention of the ²²³Ra²⁺ that may be a result of metal ion reuptake capacity of the organ. The complex status after excretion was also investigated. Urine samples from the chelator-administered group were analyzed utilizing SEC and demonstrated intact [(²²³Ra)macropa] with a matching retention time to both barium and [(²²³Ra)macropa] (see e.g., FIG. 2, FIG. 3E). Together, the rapid, intact, and kidney-specific clearance indicate a high stability of this radiocomplex in vivo.

The labeling efficiency and stability of macropa conjugates to harness ²²³Ra as a targeted alpha particle emitting moiety was tested. First, a macropa-β-alanine conjugate was synthesized and tested for metal complexation alkaline earth metals. The synthesis consisted in modifying macropa with an amine in para of one picolinic acid arm, and then converting the functional group into an isothiocyanate. The chelator was conjugated via a thiourea bridge with the N-side of β-alanine. The derived macropa-β-alanine was synthesized with 38% yield and >95% purity.

The crystal structure of [(Ba)macropa]-β-alanine was isolated in DMSO and displayed the rational feature of an integral metal complex. Similar to [(Ba)macropa], [(Ba)macropa]-β-alanine displayed six Ba—O coordinations with average bond lengths of 2.849±0.074 Å and four other Ba—N bonds average length 2.962±0.032 Å. A total of 10 coordinations were involved in barium complexation. No differences in bond lengths were noted when compared with macropa. Further comparative potentiometric titrations of macropa-β-alanine with Ca, Sr, and Ba reported the highest affinity for Ba. This reinforces the hypothesis of macropa-β-alanine may coordinate ²²³Ra in a similar fashion to the chelate itself.

Macropa-β-alanine was labeled with ²²³Ra in identical conditions to the macropa, described herein, resulting in a radiolabeling efficiency >90% (n=5; SI). As described herein, the stability of the conjugate under a serum protein challenge in vitro was tested, with a recovery of >75% of the initial activity chelated after 12 days of incubation. Low counts of ²²³Ra (≃25% of initial content) were detected in the region of protein fractions (10-15 mL). Previously, no direct affinity of free radium was observed with proteins (see e.g., FIG. 3B) suggesting here that the labeled amino acid has some affinity to bind protein.

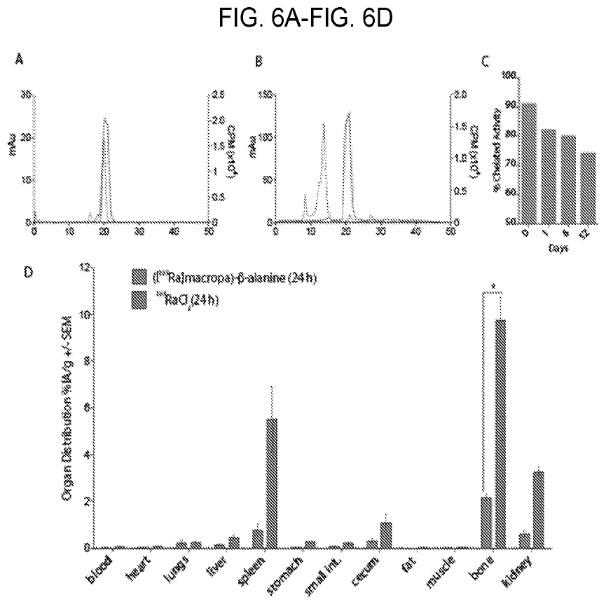

The organ distribution at 24 h p.i. (see e.g., FIG. 6D) confirmed the presence of intact ²²³Ra-labeled macropa-β-alanine in circulation with significant differences in bone uptake 2.69±0.24% IA/g versus 9.7±1.66% for ²²³RaCl₂ (P=0.0027). There was approximately 5-fold lower splenic uptake, at 0.95±0.5% IA/g [(²²³Ra)macropa-β-alanine] in comparison to 5.52+/−2.3% IA/g for the control group. The decreased kidney uptake is likely explained by earlier elevated clearance of the radiolabeled amino acid. Similarly to [(²²³Ra)macropa], major differences in biological profiles were observed as compared to ²²³RaCl₂. It was noted that the overall organ retention of ²²³Ra was lower for β-alanine than the control group; suggesting a greater clearance of ²²³Ra as well as reduced bone uptake.

[(²²³Ra)macropa]-β-alanine was observed to be stable in vitro and in vivo, this observation opens the path forward of a targeting strategy of ²²³Ra to sites of disease for targeted radioisotopic therapy.

In conclusion, it has been demonstrated herein, for the first time, the robust, rapid, and stable aqueous chelation of radium. Upon formation, the [(²²³Ra)macropa] demonstrates a high degree of stability long-term in vitro under physiological conditions and decomplexation of the ion was not detected in animal models used to assess in vivo biodistribution. The rapidly passed small molecule chelate was further excreted intact. Likewise, an amino acid conjugate, [(²²³Ra)macropa]-β-alanine, was successfully synthesized and evaluated. Other conjugates demonstrated less adequate in vivo properties. Ongoing work is focused on the role of bifunctional strategies to enable targeted alpha particle therapy constructs with the ²²³Ra-macropa system. However, by virtue of the results demonstrated herein which stand out among other approaches that have attempted to achieve radium chelation, this abundant and clinically validated alpha particle emitter may serve as the platform for novel cancer therapies.

### Example 2: Ion Channel Blockade of ²¹³Ra Gut Uptake

The following example describes the use of an ion channel modulating agent to block off-target uptake of the radiopharmaceutical ²²³Ra in the intestine.

Ionizing Radiation in Nuclear Medicine and Cancer Therapy

Ionizing radiation is commonly used in nuclear medicine and cancer therapy. The harmful effects of X-rays and gamma rays have been well-documented and preceded discovery of polonium and radium. For example, the observation that direct application of radium produced the ‘Becquerel burn’ (burns to the skin) was noted at the turn of the century. Radiopharmaceuticals are another source of ionizing radiation. Radiopharmaceuticals are agents that contain radioactive isotopes and are can be used for cancer treatment or diagnosis purposes in conjunction with medical imaging. FDA approved radiopharmaceuticals include sodium iodide (Na¹³¹I), phosphorous-32 (³²P), samarium-153 lexidronam, ²²³RaCl₂, ⁸⁹SrCl₂, ⁹⁰Y radioembolic agents and ⁹⁰Y-ibritumomab tiuxetan.

Radiation can comprise beta particles (β⁺ or β⁻), which typically carry 0.5-1.5 MeV of energy and have long track lengths (on the scale of millimeters in biological tissues). Radiation may also comprise alpha particles (α), which are charged helium nuclei that typically carry 5-8 MeV of energy and have high linear energy transfer (LET) on the scale of μm. Both beta and alpha particles can penetrate biological tissues and cause tissue and genomic damage.

Metastatic Prostate Cancer

Prostate cancer (PCa) is the most common cancer among men, and the leading cause of male cancer-related mortality after melanoma. >29,000 men succumb to PCa per year in the US. PCa is driven by the androgen receptor and this therapeutic target is the most common target after extraprostatic extension of disease. Inevitably, PCa can progress to a lethal castrate resistant status, with frequent metastatic colonization of the skeleton.

Bone metastases cause pain, loss of mobility, fracture and degrade quality of life; eventually displacing the hematological compartment. In the metastatic context, radiation is used sparingly. However, cellular, and ideally nuclear, delivery of particle emitting radiation have significant palliative and anti-neoplastic effects.

Radium-223 Dichloride (²²³RaCl₂)

Radium-223 Dichloride (²²³RaCl₂) is a bone-seeking alpha particle emitting radionuclide with approximately 5.78 MeV of decay energy and a tissue track length of 45.85 μm. ²²³RaCl₂ is currently approved for metastatic castration-resistant prostate cancer (mCRPC) without visceral metastases). Treatment with ²²³RaCl₂ has been shown to significantly increase median survival in mCRPC patients compared to control patients (14.9 months vs. 11.9 months).

A whole-body distribution of ²²³RaCl₂ shows that the primary site of alpha particle uptake is the bone; however, a significant amount is also taken up by the intestine, which is undesirable as this uptake could contribute unnecessary toxicity to intestinal cells. The intestinal uptake is significant multiple days after the ²²³RaCl₂ has been administered to the subject.

Mouse Models

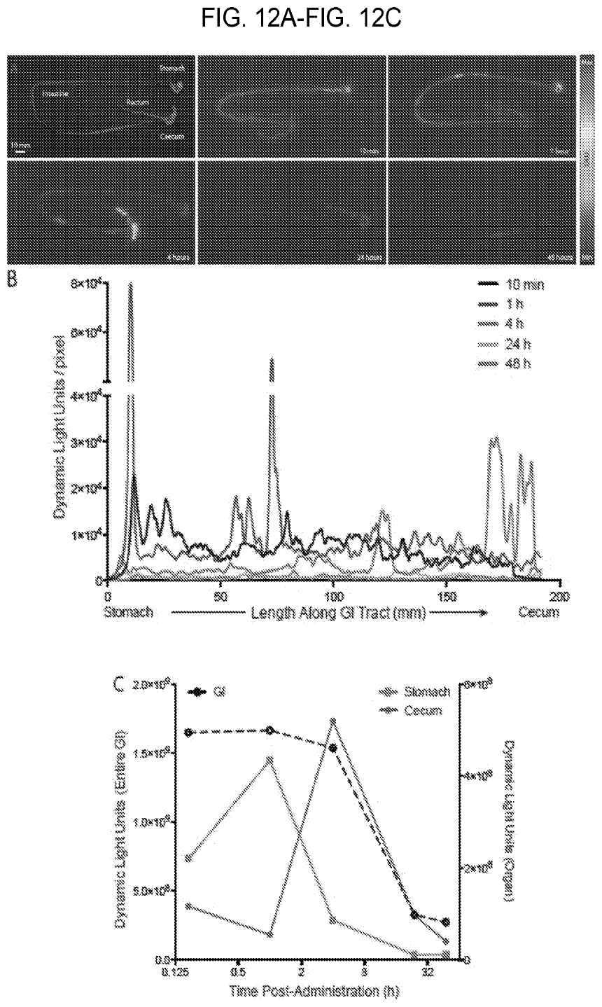

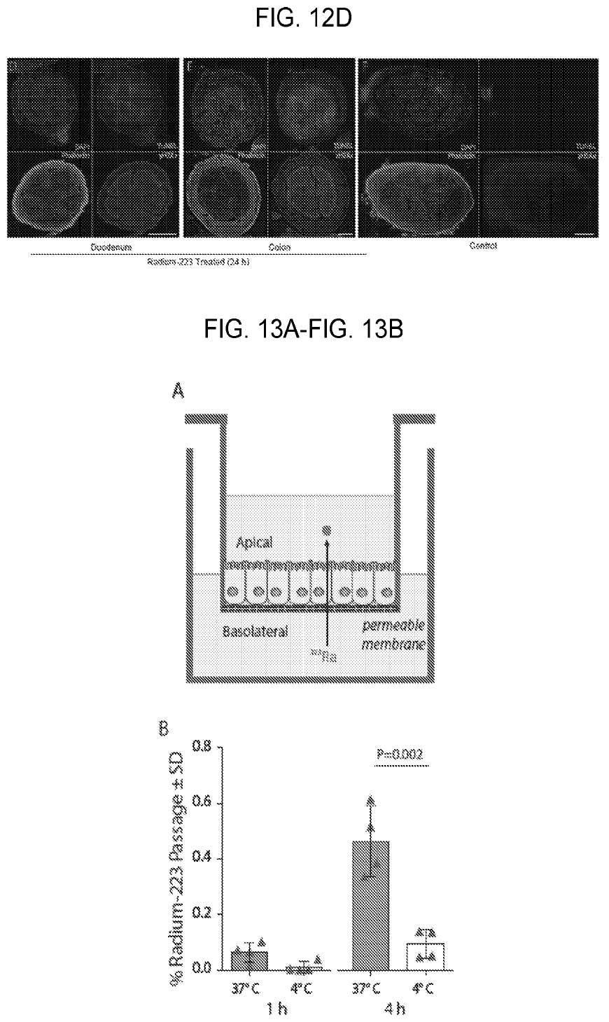

It would be preferable to use animal models to more easily study the distribution of ²²³RaCl₂ and methods and compositions of modulating it. However, first it should be confirmed that animal models are relevant. In other words, it should be determined whether the organ distribution of uptake matches what is observed in humans and whether there is any differential microdistribution. Previously, 50 kBq/kg of intravenous ²²³RaCl₂ was administered to skeletally mature mice at least 14 weeks of age and whole-body distribution of ²²³RaCl₂ was measured. It was observed that like humans, ²²³RaCl₂ was preferentially taken up by the bone (e.g., tibia, femur, and vertebrae). While there was preferential uptake in the small intestine 1 hour after administration, this was greatly reduced after 4 hours. The rapid accumulation of ²²³Ra into the gut suggests that the uptake is dominated by an active process; subsequent clearance may be dependent on bowel motility. Small bowel and stomach accumulation are observed within minutes ²²³RaCl₂ administration to mice (see e.g., FIG. 12A and FIG. 12C).

Clinical Consequences of Gut Uptake

There are multiple clinical consequences of off-target ²²³RaCl₂ uptake into the gut or intestine. Firstly, a subset of patients reports gastrointestinal distress, which can lead to treatment cessation. Secondly, the off-target uptake results in half of the administered dose failing to target the disease site. In mice, ²²³RaCl₂ uptake in the intestine results in increased cell apoptosis and DNA damage in the cells of the colon and duodenum (see e.g., FIG. 12D).

Inhibition of ²²³Ra Transport

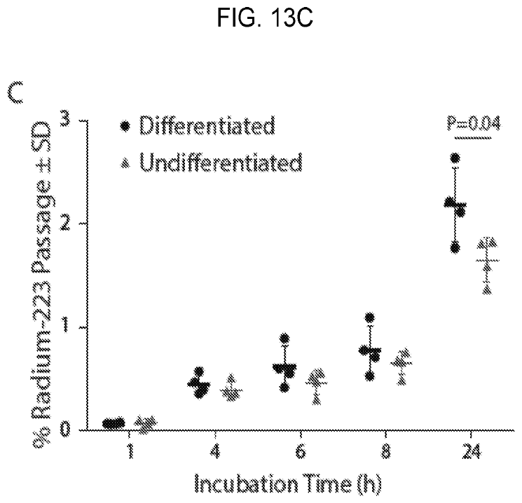

The study described herein sought to inhibit ²²³Ra transport into the intestine and gut. An in vitro patient-derived organoid model of functional gut epithelium was developed by plating human duodenal enteroid monolayers on a permeable trans-well scaffold. This enteroid recapitulates a functional intestinal barrier suited to examine ²²³Ra gastrointestinal exsorption (see e.g., FIG. 13A). The enteroid was then exposed to ²²³RaCl₂. A temperature-dependent active transport of radioactivity was observed in undifferentiated enteroids (see e.g., FIG. 13B). Differentiated versus undifferentiated enteroids were simultaneously evaluated in order to identify the role of key-ion transporters involved in ²²³Ra. Differentiated duodenal enteroids demonstrated a superior capacity to transport ²²³Ra through the monolayer from basolateral to lumen compartment as compared to the undifferentiated structures (see e.g., FIG. 13C). This indicates an ion-channel specific transport of ₂₂₃Ra is mechanistically preferred in the differentiated structures.

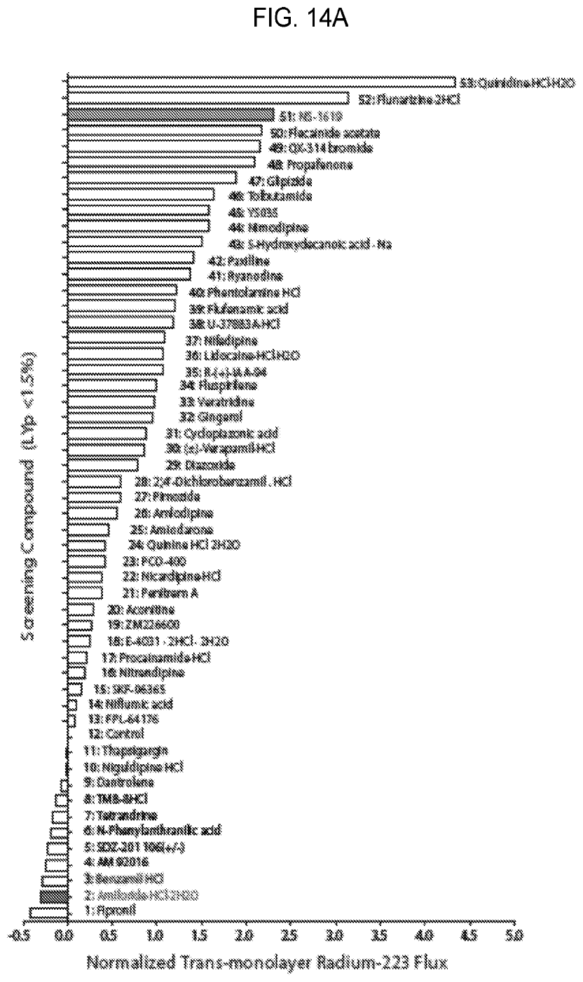

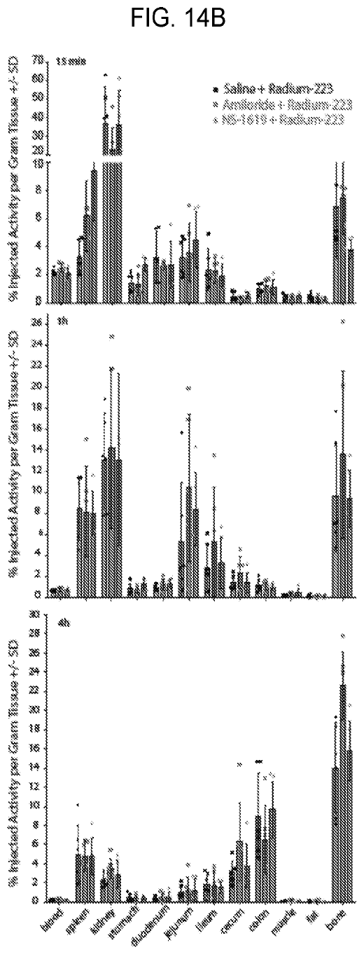

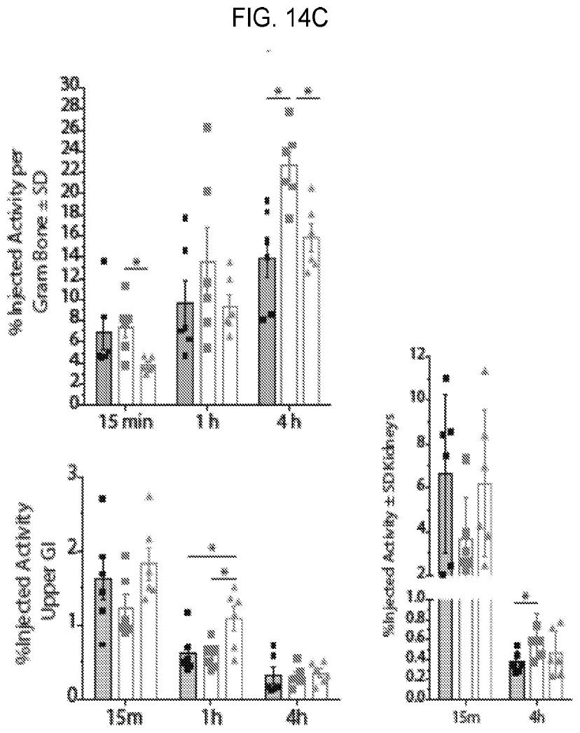

A screen was performed to identify compounds that can block the ion-channel specific transport of ₂₂₃Ra. ²²³Ra and the enteroids were co-incubated with a library of 52 ion-channel inhibitors or activators (see e.g., FIG. 14A). Amiloride and NS-1619 emerged as screen hits capable of reducing or increasing ²²³Ra transport and were selected for further in vivo validation as inhibitor and activator, respectively. It was observed that generally calcium channel inhibitors have variable efficacy (e.g., verapamil induces transport). Potassium channel inhibitors generally increase transport (e.g., NS-1619). Interestingly, thapsigargin (a membrane pore-forming toxin) does not increase transport.

Healthy male C57/Bl6 mice were randomized received a combination treatment with intraperitoneal injection of Amiloride (inhibitor, red) or NS-1619 (activator, green) or saline (control, grey) followed by a clinical dose of ²²³RaCl₂ 1 h post-drug administration. Radioactive organ distribution was then measured at 15 min, 1 h and 4 h post ²²³Ra treatment. Several organs displayed significant differences in ²²³Ra uptake over time (see e.g., FIG. 14B). Comparing bone uptake, as early as 15 min, osseous absorption is was half for the NS-1619-treated group as compared to the amiloride or the control group. Following blood and kidney clearance at 4 h p.i., ²²³Ra sequestration in bone was revealed to be over 1.5 times higher for the amiloride-treated group versus control or NS-1619.

²²³Ra activity uptake in bones at 15 min (% IA/g) was found to be twice as high in the control and amiloride-treated group compared to NS-1619 one (see e.g., FIG. 14C); this difference is further accentuated for amiloride-treated group 4 h p.i., as the bone uptake is significantly higher as compared to the two other groups. The upper GI radioactive uptake (combined % IA of stomach, duodenum, jejunum) suggests a lower radioactive content for amiloride-treated animals 15 min p.i. while the NS-1619 group shows a significantly higher ²²³Ra accumulation 1 h p.i. than the other groups. No difference was noticed 4 h p.i. Interestingly kidney uptake (% IA) shows a delayed excretion of ²²³Ra 4 h p.i. for the amiloride-treated group.

Materials and Methods

Chemicals

All the chemicals unless indicated were purchased at Sigma-Aldrich, MO. Golytely was obtained from Braintree Laboratory, MA. Immunohistological tissue stainings such as Click-iT Plus TUNEL assay were purchased from Molecular probes-Life technologies. Goat Anti-Rabbit IgG-DyLight 680 was obtained from Thermo Scientific, IL. Mounting media DAPI or TRITC (tetramethylrhodamine)-Phalloidin were purchased at Vector laboratories. Screen-well ion channel ligand library was acquired from Enzo lifesciences, NY. Microscint light scintillation cocktail was acquired from Perkin Elmer.

Isotope Production and Quality Control

²²³RaCl₂ was produced from a previously described method and generated from the parent isotope source Thorium-227 was provided by Oak Ridge, DOE. Briefly, 10-20 μCi of the parent isotope was adsorbed on a cationic polymeric resin and eluted under mild acidic conditions to isolate pure ²²³Ra-nitrates. Following multiple evaporation, the final sample was suspended in sodium citrate 0.03M and saline 150 mM, as prepared in clinical formulations. Radiochemical quality check was conducted utilizing gamma spectrometry High Purity Germanium detector, demonstrating undetectable parent breakthrough and a ²²³Ra purity >99.9%.

In Vitro Studies

Intestinal cellular models—human duodenal enteroids and caco-2 monolayers—were grown on a trans-well polymeric filter plate divided in a lower and an upper incubation compartments modeling basolateral and apical side of the epithelium, respectively. This experimental setup was utilized to identify and quantify ²²³Ra transcellular epithelial transport from basolateral (BL) membrane or blood compartment to apical (AP) membrane or lumen as a physiological mimic of ²²³Ra gastrointestinal secretion.

Human duodenal enteroids (see e.g., FIG. 13A) were reconstituted and grown from healthy patient-derived cellular crypts tissue samples. Both differentiated (DF) and undifferentiated (UDF) conditions were tested. DF monolayers were induced by removal of Wnt3A and R-spondin factors from the incubation media, as described previously.

DF or UDF duodenal enteroids were set and grown on 24-transwell plates for 2 weeks until cell confluency was reached, leading to closed monolayer membranes. Membrane integrities incubation were checked, before and after ²²³RaCl₂ treatment, measuring surface tension TEER in Ω/m (transepithelial electrical resistance) utilizing an ohmmeter. 50 nCi (1.85 KBq) was added in the basolateral compartment and incubated at 37° C., 5% CO2, 1 to 24 h (n=4) before well separation. Radioactive transport was counted utilizing light scintillation (LS) counter, sampling the apical compartment media in LS cocktail (10 mL) at various time points. % ²²³Ra passage was calculated as the ratio percentile of CPM apical measured over CPM initial activity added diluted in apical volume, background corrected. Temperature-dependent transport 37° C. versus 4° C. was tested on UDF enteroids (see e.g., FIG. 13B). DF versus UDF enteroids phenotypes transfer were compared, selecting monolayers with similar range of TEER (1200<TEER<1800 D/m) (see e.g., FIG. 13C).

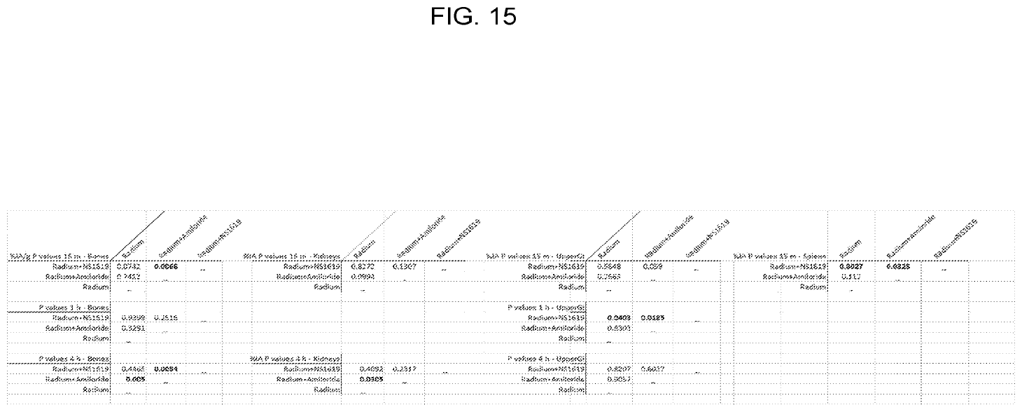

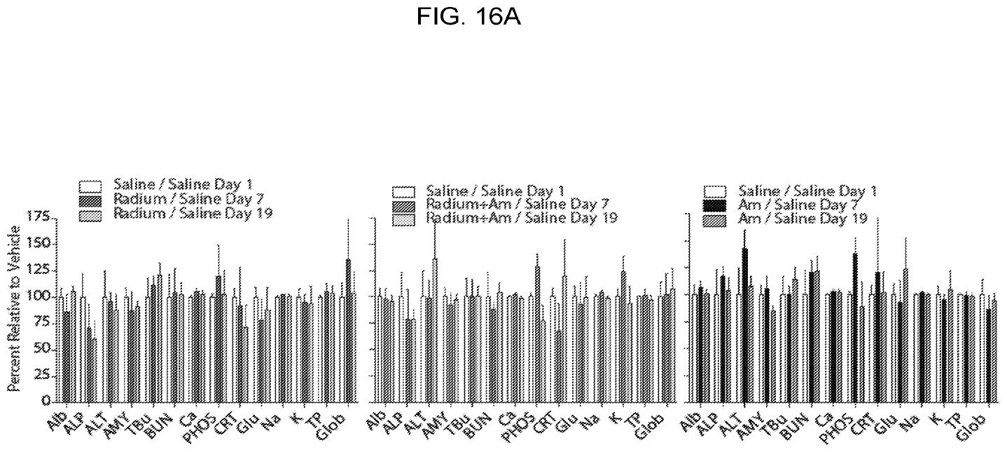

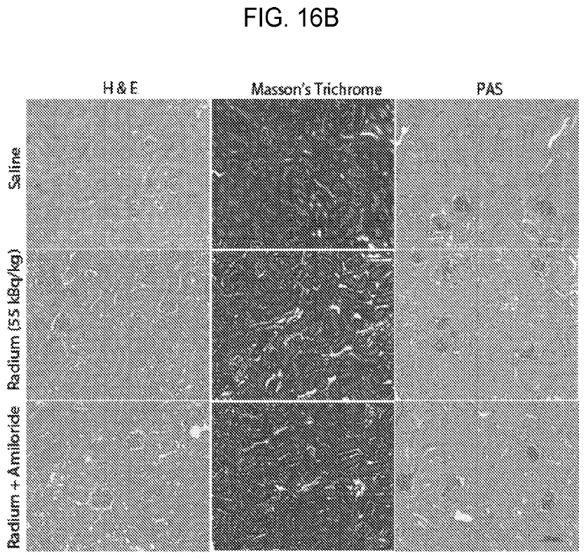

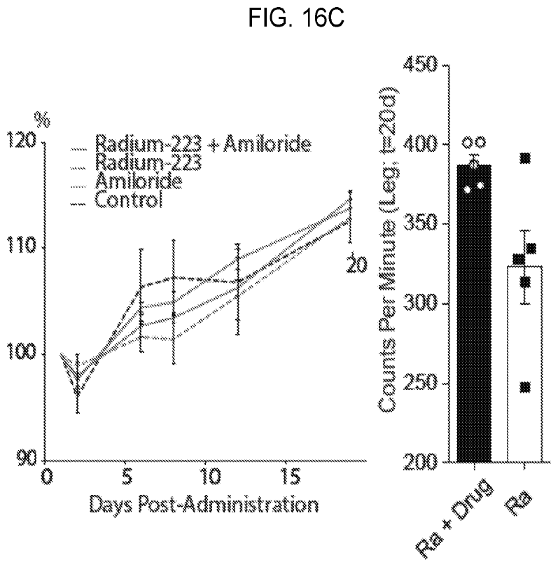

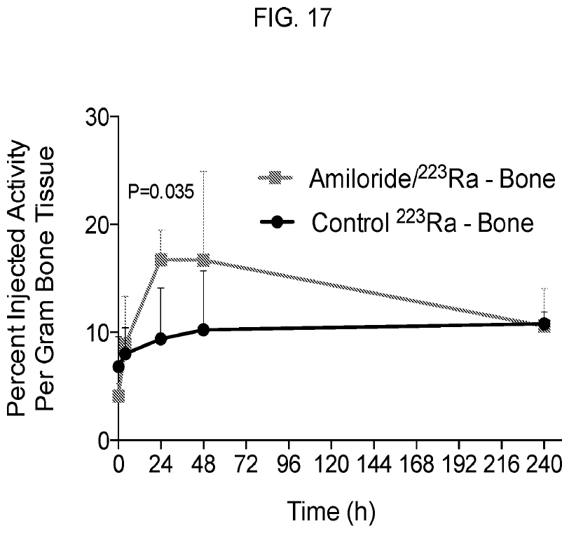

Caco-2BBe1 (brush border expressing clone of parent Caco-2 line) monolayers, modelling colon tissue, were used. Caco-2 monolayers were utilized for ion-channel ligand library screening (52 compounds) testing epithelial ion-transport activation or blocking of radioactive transfer (see e.g., FIG. 15A). Caco-2 cells were seeded 15 K cells/well in 96 trans-well filter plate and assayed 10 days post-seeding. Similarly to the enteroids assay, the efficiency of ²²³Ra transfer from BL to AP compartment was evaluated, while specifically examining ion-transport modulation. The screening material was sampled at 20 μM per BL well (n=3/molecules). 2 h following drug treatment, 3 nCi (0.1 KBq) of ²²³RaCl₂ was added in the same compartment. The incubation was stopped 4 h post ²²³RaCl₂ treatment, separating AP from BL well.

The monolayers integrity was monitored using a lucifer yellow permeability assay. The fluorescent tracer was loaded in the AP compartment, and fluorescence permeability was read in the BL vial. % Lucifer Yellow Passage (LYp %) was calculated as the percentile ratio of relative Fluorescence Units-535 nm (FU) of the assayed BL over FU of complete transfer in BL well. Permeability >1.5% showed unclosed monolayers that were excluded from experiment.

Radioactive reading was accomplished sampling AP compartments on Luma plates and reading the dried luminescence using Topcount scintillation counter. A control assay (n=6)—treatment-free—tagged “12: control” (FIG. 14A), was conducted defining the radioactive flux ²²³Ra passage % occurring through cells as the baseline value 0. Averaged drug-assayed measurements (n=3) were normalized to treatment-free flux. Results were plotted as a waterfall chart: with negative values demonstrating ²²³Ra blocking and positive values showing ²²³Ra transport activation through monolayers.

In Vivo Experiments

All animal experiments were conducted in accordance with the institutional animal care and use protocol of the Johns Hopkins University School of Medicine and Washington University School of Medicine as well as conformed to the Guide for the Care and Use of Laboratory Animal (8^{th} Ed. National Research Council of the National Academies).

Male C57Bl/6 aged 10 weeks (n=6 per treated group) were administered intraperitoneally Amiloride (12.5 mg/kg-150 uL 2% v/v DMSO) or NS-1619 (1 mg/kg-150 uL 2% v/v DMSO) or saline 1 h prior ²²³RaCl₂ i.v. administration (3.7 KBq/100 uL buffer). Animals were kept awake between each injection. 15 min; 1 h and 4 h post ²²³Ra administration: blood; spleen; kidney; stomach; duodenum; jejunum; ileum; cecum; colon; muscle; fat and bone tibia were harvested, weighed and gamma counted. Gamma counting was conducting utilizing a Perkin Elmer Wizard2 counter with readings of energies set 150-350 KeV, with 10 min reading for each sample. Standard corresponding to 10% of injected activity were measured as well; allowing for the calculation of % Injected activity (% IA) normalized to tissue weight (% IA/g).

In another experiment, mice (n=5) were randomized in 4 cohorts treated with Radium-223 alone (3.7 KBq/100 uL); or Amiloride alone (12.5 mg/kg i.p.); or combination Amiloride+Radium-223 or saline (control). The animals were maintained for 20 days with food and water and submitted to blood chemistry analysis (Abaxis Vscan 2) at day 1; 7 and 19 d post-treatment. Weight was monitored for each cohort over 20 days. At the end of the study (day 20 post-treatment), animals were sacrificed and whole limbs and kidneys were harvested for gamma counting and histopathological assessments, respectively.

Autoradiography Gastrointestinal Tract

Male C57Bl/6 aged 43 weeks old were retro-orbitally administered with 250 KBq/kg dissolved in 100 μL of buffered ²²³Ra. Animals were sacrificed at 10 min; 1 h; 4 h; 24; 48 h post ²²³Ra administration. The whole gastrointestinal tracts from stomach to rectum were rapidly harvested and placed directly on plastic wraps set over autoradiographic phosphor screens for 90 min exposure in the dark. The autoradiographic prints were read on Cyclone Phosphor Imager (Packard), 300 DPI confocal laser stimulation; 16-bit ADC; utilizing FIJI processing with median filter. For FIJI readings of the autoradiographic files, data were converted to 32-bit format utilizing “square and scale n16” plug in. Radioactive signal intensity was translated into dynamic light units (see e.g., FIG. 12A). Signal intensity was plotted over the GI tract length from the stomach (start=0) to the cecum (˜200 mm) comparing various time points (see e.g., FIG. 12B). Results were comprised as well into profiles curves per organ region of interests including: stomach, cecum or the whole intestinal tract (see e.g., FIG. 12C).

Animal Treatments and Immunohistological Stainings

Male C57Bl/6 aged 20-30 weeks old were retro-orbitally administered with 250 KBq/kg dissolved in 100 μL of buffered ²²³Ra Dichloride. Animals were fasted and supplemented with Golytely (24 g/350 mL) 2 h prior injections and sacrificed 24 h post administration. Sections (1 cm) of each gastrointestinal region from the duodenum, jejunum, ileum to the colon were rapidly harvested. GI specimen were set onto previously partially solidified OCT gel block, then fully coated with OCT and flash frozen. Gastrointestinal transversal sectioning was conducted utilizing Leica microtome, 10 μm sections. Fresh frozen sections were collected on tape and stored at −20° C.

Gastrointestinal tissue sections were fixed immerging tapes in 10% paraformaldehyde (PFA) for 15 min followed by a washing with PBS 1×. Following fixation, tissues were mounted directly with DAPI and/or TRITC-Phalloidin mounting media under cover slip ready for imaging.

For TUNEL staining, tissues were permeabilized, treated with 0.25% Triton x-100 for 15 min and washed with PBS. A second fixations was proceeded using 10% PFA and washed with deionized water. Tissues were then reacted using buffer reaction mixtures from Click-iT Plus TUNEL assay. Positive control were conducted, treated similarly with an additional DNAase treatment. Final counterstaining was conducted using Hoechst33342 (1:5000) before mounting taped tissue specimen under cover slip with 30% aqueous glycerol.

For γH2AX staining, tissues were fixed and submitted to blocking serum reagent, 1 h at room temperature. The primary antibody (γH2AX, rabbit antibody 1 mg/mL-1:50) was applied overnight at 4° C. in humidity chamber. Tissues were then washed and prepared for reaction with the secondary antibody goat anti-rabbit IgG Alexa Fluor 488 (1:200) 1 h at 4° C. in humidity chamber. The resulting samples were washed, counterstained using Hoechst 3342 and mounted under cover slip with glycerol 30%.

Kidneys were collected 20 days post-treatment and the tissues were fixed in formalin for 72 h then transferred to saline. Tissues were processed by dehydration, paraffin embedding, and sectioning at 4 μm. Adjacent sections were stained with H&E, PAS and Masson's trichrome.

Statistics

All P values were calculated with unpaired two-tailed t-test with equal Standard Deviation (SD), utilizing Graphpad, Prism software. Significant differences were expressed at P<0.05 and P<0.1 for in vitro (n=4) and in vivo work (n=6).

1. A method for minimizing or mitigating exposure of bodily retention of alpha particle-emitting radium (Ra) isotope in a subject comprising:
administering a therapeutically effective amount of a pharmaceutical composition comprising a macrocyclic ligand selected from a macropa and a macropa derivative to the subject, wherein the macrocyclic ligand is chelated to an alpha particle-emitting radium (Ra) isotope, resulting in a macrocyclic ligand-radium complex.

2. (canceled)

3. The method of claim 1, wherein the macrocyclic ligand-radium complex has reduced bone accumulation compared with bone accumulation for a non-chelated radium.

4. The method of claim 1, wherein the subject has cancer or metastatic cancer.

5. The method of claim 1, wherein the macropa derivative is macropa-NCS.

6. The method of claim 1, wherein the alpha particle-emitting Ra isotope is selected from ²²³Ra, ²²⁴Ra, ²²⁵Ra, or ²²⁶Ra.

7. The method of claim 1, wherein the macrocyclic ligand-radium complex is (²²³Ra)macropa of formula

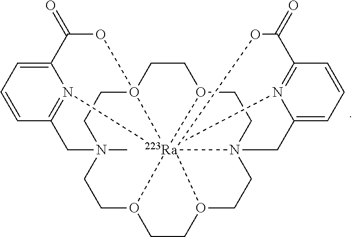

8. The method of claim 1, wherein the macrocyclic ligand is conjugated to a cancer-targeting agent.

9. The method of claim 8, wherein the cancer-targeting agent is a monoclonal antibody, a functional fragment of an antibody, a recombinant protein, a single chain variable fragment (scFv), or a peptide.

10. The method of claim 1, wherein the macrocyclic ligand-radium complex does not target bone or bone tissue.

11. A pharmaceutical composition comprising a macrocyclic ligand selected from a macropa and a macropa derivative, wherein the macrocyclic ligand is chelated to an alpha particle-emitting radium (Ra) isotope, resulting in macrocyclic ligand-radium complex.

12. The pharmaceutical composition of claim 11, wherein the macropa derivative is macropa-NCS.

13. The pharmaceutical composition of claim 11, wherein the alpha particle-emitting Ra isotope is selected from ²²³Ra, ²²⁴Ra, ²²⁵Ra, or ²²⁶Ra.

14. The pharmaceutical composition of claim 11, wherein the macrocyclic ligand-radium complex is (²²³Ra)macropa of formula

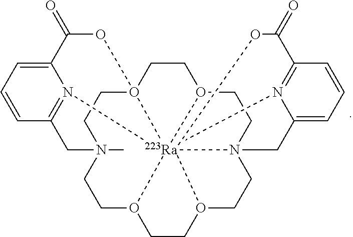

15. The pharmaceutical composition of claim 11, wherein the macrocyclic ligand is conjugated to a cancer-targeting agent.

16. The pharmaceutical composition of claim 15, wherein the cancer-targeting agent is a monoclonal antibody, a functional fragment of an antibody, a recombinant protein, a single chain variable fragment (scFv), or a peptide.

17. A method of making an alpha particle-emitting therapeutic agent comprising an alpha particle-emitting isotope complexed with a macrocyclic ligand comprising combining a macrocyclic ligand selected from a macropa and a macropa derivative, Ra(NO₃), RaCl₂, and trimethyl ammonium acetate, wherein the alpha particle-emitting isotope is a radium isotope and the macrocyclic ligand, Ra(NO₃), RaCl₂, and trimethyl ammonium acetate are combined for a period of time sufficient to result in a macrocyclic ligand-radium complex.

18. The method of claim 17, wherein the macropa derivative is macropa-NCS.

19. The method of claim 17, wherein the Ra isotope is selected from ²²³Ra, ²²⁴Ra, ²²⁵Ra, or ²²⁶Ra.

20. The method of claim 17, comprising conjugating a cancer-targeting agent to the macrocyclic ligand.

21. The method of claim 20, wherein the cancer-targeting agent is a monoclonal antibody, a functional fragment of an antibody, a recombinant protein, a single chain variable fragment (scFv), or a peptide.

22. The method of claim 17, wherein the combining is performed at about 25° C. and a pH of about 6, for an amount of time sufficient to form a macrocyclic ligand-radium complex, wherein the amount of time sufficient to form a macrocyclic ligand-radium complex is less than an hour or between about 5 minutes and about 2 hours.

23. The method of claim 17, wherein the macrocyclic ligand-radium complex does not target bone or bone tissue.

24. The method of claim 17, wherein the macrocyclic ligand is conjugated to a cancer-targeting agent.

25. The method of claim 24, wherein the cancer-targeting agent is a monoclonal antibody, a functional fragment of an antibody, a recombinant protein, a single chain variable fragment (scFv), or a peptide.

26. A method of reducing off-target uptake of a radiopharmaceutical or improving radiopharmaceutical uptake or localization to a target tissue in a subject in need thereof comprising
administering to the subject an ion channel blocking agent in an amount effective to
(i) reduce off-target tissue localization of the radiopharmaceutical; or
(ii) increase uptake, targeting, or localization of a radiopharmaceutical in the target tissue compared to targeting or localization of the radiopharmaceutical in the target tissue without the ion channel blocking agent.

27. (canceled)

28. The method of claim 26, wherein the target tissue is a bone metastasis.

29. The method of claim 26, wherein the radiopharmaceutical is a radiopharmaceutical having chemical similarity to calcium or binds to a calcium channel.

30. The method of claim 26, wherein the radiopharmaceutical comprises ²²³Ra.

31. The method of claim 30, wherein the radiopharmaceutical is ²²³RaCl₂.

32. The method of claim 26, wherein the ion channel blocking agent is a calcium channel inhibitor or blocking agent.

33. The method of claim 26, wherein the ion channel blocking agent selected from one or more of the group consisting of: Fipronil; Amiloride; Benzamil HCl; AM 92016; SDZ-201 106(+/−); N-Phenylanthranilic acid; Tetrandrine; TMB-8HCl; Dantrolene; Niguldipine HCl; Thapsigargin; and combinations, variants, or analogues thereof.

34. The method of claim 26, wherein the ion channel blocking agent is a calcium channel inhibitor selected from amiloride.

35. The method of claim 32, wherein the calcium channel inhibitor reduces gastrointestinal uptake of the radiopharmaceutical compared to gastrointestinal uptake of the radiopharmaceutical if no calcium channel inhibitor was administered.

36. The method of claim 26, wherein the ion channel inhibiting agent is a radiopharmaceutical transport inhibiting agent increases radiopharmaceutical uptake in bone compared to the uptake in bone of a subject not treated with the calcium channel inhibitor.
